SEQUENCE LISTING (61 sequences)

SEQ ID NO: 1 - protein, 250 aa, Homo sapiens
GAMGSMGDEDWEAEINPHMSSYVPIFEKDRYSGENGDNFNRTPASSSEMDDGPSRRDHFMKSGFASGRNFGNRDAGECNKRDNTSTMGGFGVGKSFGNRGFSNSRFEDGDSSGFWRESSNDCEDNPTRNRGFSKRGGYRDGNNSEASGPYRRGGRGSFRGCRGGFGLGSPNNDLDPDECMQRTGGLFGSRRPVLSGTGNGDTSQSRSGSGSERGGYKGLNEEVITGSGKNSWKSEAEGGESSDTQGPKVT

SEQ ID NO: 2 - protein, 239 aa, Artificial Sequence
  GFP synthetic construct
MHHHHHHMSKGEELFTGVVPILVELDGDVNGHKFSVRGEGEGDATNGKLTLKFICTTGKLPVPWPTLVTTLGYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHG

SEQ ID NO: 3 - protein, 245 aa, Artificial Sequence
  GFP synthetic construct
MHHHHHHMSKGERLFTGVVPILVELDGDVNGHKFSVRGEGEGDATRGKLTLKFICTTGKLPVPWPTLVTTLTYGVQCFSRYPKHMKRHDFFKSAMPEGYVQERTISFKKDGTYKTRAEVKFEGRTLVNRIELKGRDFKEKGNILGHKLEYNFNSHNVYITADKRKNGIKANFKIRHNVKDGSVQLADHYQQNTPIGRGPVLLPRNHYLSTRSALSKDPKEKRDHMVLLEFVTAAGITHGMDELYK

SEQ ID NO: 4 - protein, 245 aa, Artificial Sequence
  GFP synthetic construct
MHHHHHHMSKGEELFDGVVPILVELDGDVNGHEFSVRGEGEGDATEGELTLKFICTTGELPVPWPTLVTTLTYGVQCFSDYPDHMDQHDFFKSAMPEGYVQERTISFKDDGTYKTRAEVKFEGDTLVNRIELKGIDFKEDGNILGHKLEYNFNSHDVYITADKQENGIKAEFEIRHNVEDGSVQLADHYQQNTPIGDGPVLLPDDHYLSTESALSKDPNEDRDHMVLLEFVTAAGIDHGMDELYK

SEQ ID NO: 5 - protein, 474 aa, Artificial Sequence
  Fusion protein
GAMGSMGDEDWEAEINPHMSSYVPIFEKDRYSGENGDNFNRTPASSSEMDDGPSRRDHFMKSGFASGRNFGNRDAGECNKRDNTSTMGGFGVGKSFGNRGFSNSRFEDGDSSGFWRESSNDCEDNPTRNRGFSKRGGYRDGNNSEASGPYRRGGRGSFRGCRGGFGLGSPNNDLDPDECMQRTGGLFGSRRPVLSGTGNGDTSQSRSGSGSERGGYKGLNEEVITGSGKNSWKSEAEGGESVDMVSKGEELFTGVVPILVELDGDVNGHKFSVSGEGEGDATYGKLTLKFICTTGKLPVPWPTLVTTFGYGLMCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSKLSKDPNEKRDHMVLLEFVTAAGIT

SEQ ID NO: 6 - protein, 535 aa, Artificial Sequence
  Fusion protein
GAMGSNMGDEDWEAEINPHMSSYVPIFEKDRYSGENGDNFNRTPASSSEMDDGPSRRDHFMKSGFASGRNFGNRDAGECNKRDNTSTMGGFGVGKSFGNRGFSNSRFEDGDSSGFWRESSNDCEDNPTRNRGFSKRGGYRDGNNSEASGPYRRGGRGSFRGCRGGFGLGSPNNDLDPDECMQRTGGLFGSRRPVLSGTGNGDTSQSRSGSGSERGGYKGLNEEVITGSGKNSWKSEAEGGESSDTQGPKVTLQMVSKGEELFTGVVPILVELDGDVNGHKFSVSGEGEGDATYGKLTLKFICTTGKLPVPWPTLVTTFGYGLMCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSKLSKDPNEKRDHMVLLEFVTAAGITLEFSTYIPGFSGSTRGNVFASVDTRKGKSTLNTAGFSSSQAPNPVDDESWD

SEQ ID NO: 7 - protein, 535 aa, Artificial Sequence
  Fusion protein
GAMGSNMGDEDWEAEINPHMSSYVPIFEKDRYSGENGDNFNRTPASSSEMDDGPSRRDHFMKSGAASGRNAGNRDAGECNKRDNTSTMGGAGVGKSAGNRGASNSRFEDGDSSGFWRESSNDCEDNPTRNRGASKRGGYRDGNNSEASGPYRRGGRGSARGCRGGAGLGSPNNDLDPDECMQRTGGLAGSRRPVLSGTGNGDTSQSRSGSGSERGGYKGLNEEVITGSGKNSWKSEAEGGESSDTQGPKVTLQMVSKGEELFTGVVPILVELDGDVNGHKFSVSGEGEGDATYGKLTLKFICTTGKLPVPWPTLVTTFGYGLMCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSKLSKDPNEKRDHMVLLEFVTAAGITLEASTYIPGASGSTRGNVFASVDTRKGKSTLNTAGFSSSQAPNPVDDESWD

SEQ ID NO: 8 - protein, 261 aa, Artificial Sequence
  YFP synthetic construct
GAMGSMVSKGEELFTGVVPILVELDGDVNGHKFSVSGEGEGDATYGKLTLKFICTTGKLPVPWPTLVTTFGYGLMCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSKLSKDPNEKRDHMVLLEFVTAAGITLERPLSNLEPAVSRHAVPSFKLSNN

SEQ ID NO: 9 - protein, 405 aa, Artificial Sequence
  Fusion protein
MFGRDPFDSLFERMFKEFFATPMTGTTMIQSSTGIQISGKGFMPISIIEGDQHIKVIAWLPGVNKEDIILNAVGDTLEIRAKRSPLMITESERIIYSEIPEEEEIYRTIKLPATVKEENASAKFENGVLSVILPKAESSIKKGINIEASGENLYFQSLSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKKGLEHHHHHH

SEQ ID NO: 10 - protein, 433 aa, Artificial Sequence
  Fusion protein
MDIAIHHPWIRRPFFPFHSPSRLFDQFFGEHLLESDLFPTSTSLSPFYLRPPSFLRAPSWFDTGLSEMRLEKDRFSVNLDVKHFSPEELKVKVLGDVIEVHGKHEERQDEHGFISREFHRKYRIPADVDPLTITSSLSSDGVLTVNGPRKQVSGPERTIPITREEKPAVTAAPKKASGENLYFQSLSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKKGLEHHHHHH

SEQ ID NO: 11 - protein, 350 aa, Artificial Sequence
  Fusion protein
CFPFVTAAGITGAMGSMTGRARARARGRARGQETAQLVGSTASQQPGYIQPRPQPPPAEGELFGRGRQRGTAGGTAKSQGLQISAGFQELSLAERGGRRRDFHDLGVNTRQNLDHVKESKTGSSGVDMVSKGEELFTGVVPILVELDGDVNGHKFSVSGEGEGDATYGKLTLKFICTTGKLPVPWPTLVTTLTWGVQCFARYPDHMKQHDFFKSAMPEGYVQERTIFFKDDGNYKTRAEVKFEGDTLVNRIELKGIDFKEDGNILGHKLEYNAISDNVYITADKQKNGIKANFKIRHNIEDGSVQLADHYQQNTPIGDGPVLLPDNHYLSTQSKLSKDPNEKRDHMVLLE

SEQ ID NO: 12 - DNA, 12 bp, Artificial Sequence
  Oligonucleotide
actgactgactg

SEQ ID NO: 13 - DNA, 12 bp, Artificial Sequence
  Oligonucleotide
cagtcagtcagt

SEQ ID NO: 14 - DNA, 16 bp, Artificial Sequence
  Oligonucleotide
actgactgactgactg

SEQ ID NO: 15 - DNA, 16 bp, Artificial Sequence
  Oligonucleotide
cagtcagtcagtcagt

SEQ ID NO: 16 - DNA, 20 bp, Artificial Sequence
  Oligonucleotide
actgactgactgactgactg

SEQ ID NO: 17 - DNA, 20 bp, Artificial Sequence
  Oligonucleotide
cagtcagtcagtcagtcagt

SEQ ID NO: 18 - protein, 20 aa, Artificial Sequence
  Oligonucleotide
ACTGACTGACTGACTGACTG

SEQ ID NO: 19 - DNA, 24 bp, Artificial Sequence
  Oligonucleotide
cagtcagtcagtcagtcagtcagt

SEQ ID NO: 20 - DNA, 28 bp, Artificial Sequence
  Oligonucleotide
actgactgactgactgactgactgactg

SEQ ID NO: 21 - DNA, 28 bp, Artificial Sequence
  Oligonucleotide
cagtcagtcagtcagtcagtcagtcagt

SEQ ID NO: 22 - DNA, 32 bp, Artificial Sequence
  Oligonucleotide
actgactgactgactgactgactgactgactg

SEQ ID NO: 23 - DNA, 32 bp, Artificial Sequence
  Oligonucleotide
cagtcagtcagtcagtcagtcagtcagtcagt

SEQ ID NO: 24 - DNA, 36 bp, Artificial Sequence
  Oligonucleotide
actgactgactgactgactgactgactgactgactg

SEQ ID NO: 25 - DNA, 36 bp, Artificial Sequence
  Oligonucleotide
cagtcagtcagtcagtcagtcagtcagtcagtcagt

SEQ ID NO: 26 - DNA, 40 bp, Artificial Sequence
  Oligonucleotide
actgactgactgactgactgactgactgactgactgactg

SEQ ID NO: 27 - DNA, 40 bp, Artificial Sequence
  Olignucleotide
cagtcagtcagtcagtcagtcagtcagtcagtcagtcagt

SEQ ID NO: 28 - DNA, 12 bp, Artificial Sequence
  Oligonucleotide
actgactgactg

SEQ ID NO: 29 - DNA, 12 bp, Artificial Sequence
  Oligonucleotide
actgactgactg

SEQ ID NO: 30 - DNA, 16 bp, Artificial Sequence
  Oligonucleotide
actgactgactgactg

SEQ ID NO: 31 - DNA, 16 bp, Artificial Sequence
  Oligonucleotide
actgactgactgactg

SEQ ID NO: 32 - DNA, 20 bp, Artificial Sequence
  Oligonucleotide
actgactgactgactgactg

SEQ ID NO: 33 - DNA, 20 bp, Artificial Sequence
  Oligonucleotide
actgactgactgactgactg

SEQ ID NO: 34 - DNA, 24 bp, Artificial Sequence
  Oligonucleotide
actgactgactgactgactgactg

SEQ ID NO: 35 - DNA, 24 bp, Artificial Sequence
  Oligonucleotide
actgactgactgactgactgactg

SEQ ID NO: 36 - DNA, 28 bp, Artificial Sequence
  Oligonucleotide
actgactgactgactgactgactgactg

SEQ ID NO: 37 - DNA, 28 bp, Artificial Sequence
  Oligonucleotide
actgactgactgactgactgactgactg

SEQ ID NO: 38 - DNA, 32 bp, Artificial Sequence; identical to SEQ ID NO: 22
  Oligonucleotide

SEQ ID NO: 39 - DNA, 32 bp, Artificial Sequence; identical to SEQ ID NO: 22
  Oligonucleotide

SEQ ID NO: 40 - DNA, 36 bp, Artificial Sequence; identical to SEQ ID NO: 24
  Oligonucleotide

SEQ ID NO: 41 - DNA, 36 bp, Artificial Sequence; identical to SEQ ID NO: 24
  Oligonucleotide

SEQ ID NO: 42 - DNA, 40 bp, Artificial Sequence; identical to SEQ ID NO: 26
  Oligonucleotide

SEQ ID NO: 43 - DNA, 40 bp, Artificial Sequence; identical to SEQ ID NO: 26
  Oligonucleotide

SEQ ID NO: 44 - RNA, 12 nt, Artificial Sequence
  Oligonucleotide
acugacugacug

SEQ ID NO: 45 - RNA, 12 nt, Artificial Sequence
  Oligonucleotide
cagucagucagu

SEQ ID NO: 46 - RNA, 24 nt, Artificial Sequence
  Oligonucleotide
acugacugacugacugacugacug

SEQ ID NO: 47 - RNA, 24 nt, Artificial Sequence
  Oligonucleotide
cagucagucagucagucagucagu

SEQ ID NO: 48 - RNA, 24 nt, Artificial Sequence
  Oligonucleotide
acugacugacugacugacugacug

SEQ ID NO: 49 - DNA, 24 bp, Artificial Sequence
  Oligonucleotide
cagtcagtcagtcagtcagtcagt

SEQ ID NO: 50 - RNA, 12 nt, Artificial Sequence
  Oligonucleotide
acugacugacug

SEQ ID NO: 51 - RNA, 12 nt, Artificial Sequence
  Oligonucleotide
acugacugacug

SEQ ID NO: 52 - RNA, 24 nt, Artificial Sequence
  Oligonucleotide
acugacugacugacugacugacug

SEQ ID NO: 53 - RNA, 24 nt, Artificial Sequence
  Oligonucleotide
acugacugacugacugacugacug

SEQ ID NO: 54 - DNA, 32 bp, Artificial Sequence
  Oligonucleotide
ggcggcttttttttttttttttttttgccgcc

SEQ ID NO: 55 - RNA, 32 nt, Artificial Sequence
  Oligonucleotide
ggcggcuuuuuuuuuuuuuuuuuuuugccgcc

SEQ ID NO: 56 - DNA, 36 bp, Artificial Sequence
  Oligonucleotide
ggcggcggttttttttttttttttttttccgccgcc

SEQ ID NO: 57 - DNA, 40 bp, Artificial Sequence
  Oligonucleotide
ggcggcggcgttttttttttttttttttttcgccgccgcc

SEQ ID NO: 58 - RNA, 22 nt, Artificial Sequence
  Oligonucleotide
ugagguaguagguuguauaguu

SEQ ID NO: 59 - RNA, 28 nt, Artificial Sequence
  Oligonucleotide
uaccacuuagaacacaggaugucagcgc

SEQ ID NO: 60 - RNA, 30 nt, Artificial Sequence
  Oligonucleotide
ugugaauggaaguccaaggaucuagcaguu

SEQ ID NO: 61 - RNA, 32 nt, Artificial Sequence
  Oligonucleotide
ugaaacuccaaaguuucuccaaggcaaaagac

(19) United States
(12) Patent Application Publication
(10) Pub. No.: US 2018/0313827 A1
(43) Pub. Date: Nov. 1, 2018

(54) BIOMOLECULE SEPARATION AND MODIFICATION

(71) Applicant: OXFORD UNIVERSITY INNOVATION LTD, Oxford Oxfordshire (GB)

(72) Inventors: Andrew Baldwin, Oxford Oxfordshire (GB); Timothy Nott, Oxford Oxfordshire (GB)

(21) Appl. No.: 15/769,597

(22) PCT Filed: Oct. 18, 2016
(86) PCT No.: PCT/GB2016/053239

§ 371 Date: Apr. 19, 2018

(30) Foreign Application Priority Data
Oct. 19, 2015 (GB) 1518477.3

(51) Int. Cl. G01N 33/543; C12Q 1/6869; C07K 1/32; C07H 1/06; C07H 21/02; C07H 21/04

(52) U.S. Cl. CPC G01N 33/54313; C12Q 1/6869; C07K 1/32; C12Q 2563/116; C07H 21/02; C07H 21/04; C12Q 2563/113; C07H 1/06

(57) ABSTRACT

The invention relates to a method of selective separation of target molecules from a heterogeneous mixture of target molecules and non-target molecules in a solution, the method comprising: providing a proteinaceous phase in the solution, wherein the proteinaceous phase comprises a matrix of self-assembling proteins, and wherein the proteinaceous phase selectively absorbs the target molecules into the matrix and/or selectively excludes non-target molecules from the matrix thereby selectively isolating the target molecules from the non-target molecules in the solution. The invention further relates to alternative methods of selective separation of target molecules, methods of ion torrent sequencing, compositions, kits and uses thereof.

The present invention relates to a method of selective sequestration, separation, extraction and purification of target molecules, and associated uses, compositions and kits.

Purification of biomolecules is a key requirement for a broad range of research, diagnostic and commercial applications. Traditional approaches involve procedures that can be laborious, require expert knowledge and often entail exposing biomolecules to non-physiological conditions. Existing approaches also assume cells have to be lysed before biomolecules can be purified. In particular, the typical methods for separating nucleic acids and proteins on a preparative or analytical scale rely on lysis of the cell followed by extraction with organic solvents (e.g. phenol/chloroform) or via solid-phase extraction. Such methods are time consuming and often provide only a low yield of the target molecule to be separated. Current solvent extraction techniques can also disrupt the native structure, intermolecular interactions and post-translational modifications of the molecule to be separated. Some biomolecules are also very difficult to separate from complex mixtures and require several steps of purification.

An aim of the present invention is to provide an alternative or improved method of separation, extraction and purification of target molecules.

According to the first aspect of the invention, there is provided a method of selective separation of target molecules from a heterogeneous mixture of target molecules and non-target molecules in a solution, the method comprising:
providing a proteinaceous phase in the solution, wherein the proteinaceous phase comprises a matrix of self-assembling proteins, and
wherein the proteinaceous phase selectively absorbs the target molecules into the matrix and/or selectively excludes non-target molecules from the matrix thereby selectively isolating the target molecules from the non-target molecules in the solution.

The proteinaceous phase may be bi-phasic. For example, the self-assembling proteins of the proteinaceous phase may associate or disassociate from each other to form, re-form or disperse the proteinaceous phase depending on the solution conditions. Therefore, in one embodiment the proteinaceous phase is provided in the solution by providing the self-assembling proteins in liquid phase in the solution, and allowing or inducing phase transition by the assembly of the self-assembling proteins into a matrix to form the proteinaceous phase.

A proteinaceous phase may form a globule, herein referred to as a “proteinaceous phase globule”. The term “proteinaceous phase” or proteinaceous phase globule” may also be referred to as a “membraneless organelle”.

The invention is a novel variation of solvent extraction, where the solvent is a proteinaceous liquid phase and it is the first demonstration of solvent extraction using liquid-droplets composed of disordered proteins. An advantage of the invention lies in the ability to separate nucleic acids based on their structure (e.g. double-stranded, single-stranded or higher structure, such as hairpin nucleic acid, or protein structure) and on their length (e.g. separation of a 12mer duplex from a 24mer duplex). Another advantage is that the separation proceeds in an aqueous environment which maintains native structure, intermolecular interactions and posttranslational modifications of the target molecule to be separated. Advantageously, the proteinaceous phase globules can destabilise one of the most stable biological structures known, the DNA double helix, by up to 6 orders of magnitude while simultaneously stabilising the structures formed by single stranded nucleic acids such as regulatory RNAs. The reversibility of the phase transition allows controlled dissolution of the extraction solvent for downstream applications. Through directed mutagenesis, the extraction properties may be tuneable and evolvable, lending unprecedented control over the types of species extracted and conditions under which the proteinaceous phase is formed.

Droplets of the self-assembling protein liquid phase are held together through many weak electrostatic interactions, formed by a pattern of charged and aromatic amino acids in the protein chain. The phase transition is initiated when a mono-disperse solution of the self-assembling protein is quenched below the phase boundary by adjustment of the conditions in the solution. Such conditions may comprise adjusting one or more of temperature, ionic strength, pH and protein concentration. Once formed, the proteinaceous phase (membraneless organelle) constitutes a dense, viscous liquid with a dielectric distinct from bulk water, capable of selectively absorbing (solubilising) or excluding target molecules.

Induction of the assembly of the self-assembling protein into the proteinaceous phase may comprise the induction of a phase transition of the self-assembling protein. The induction of the phase transition into the proteinaceous phase (i.e. phase transition from the self-assembling protein dispersed in a liquid phase to the assembly into a proteinaceous phase) may comprise modulation of salt concentration and/or temperature. Additionally or alternatively, the induction of the phase transition may comprise modification of the pH.

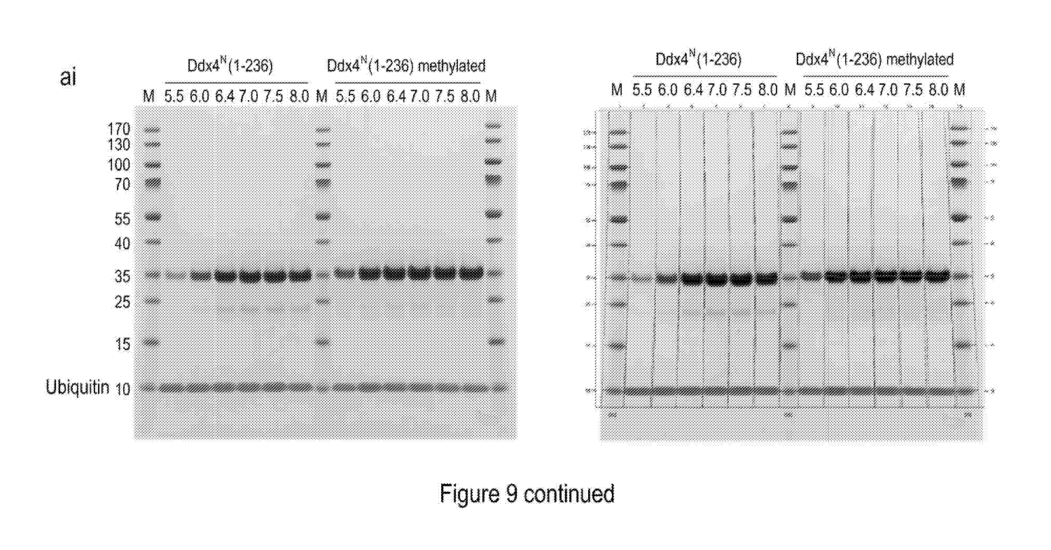

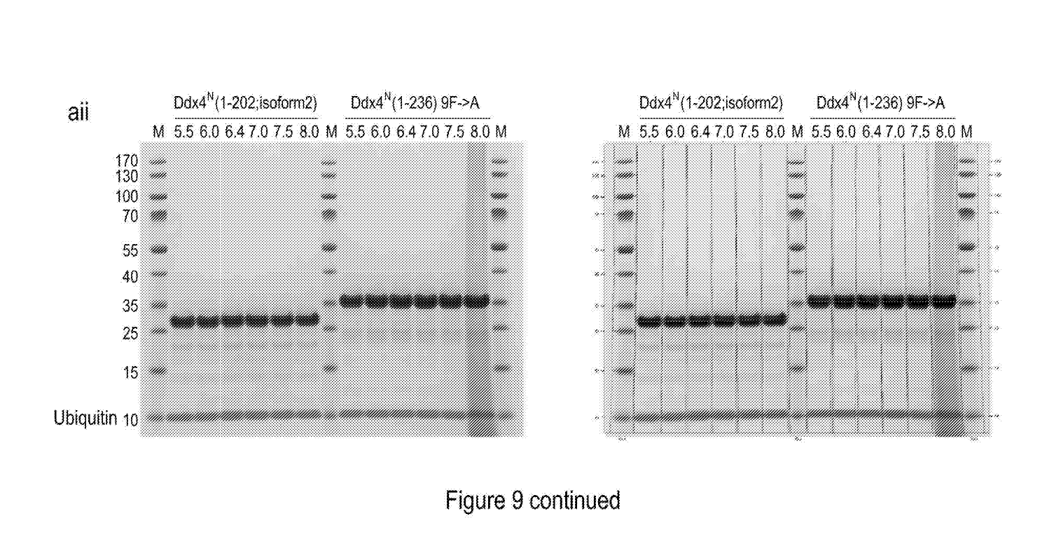

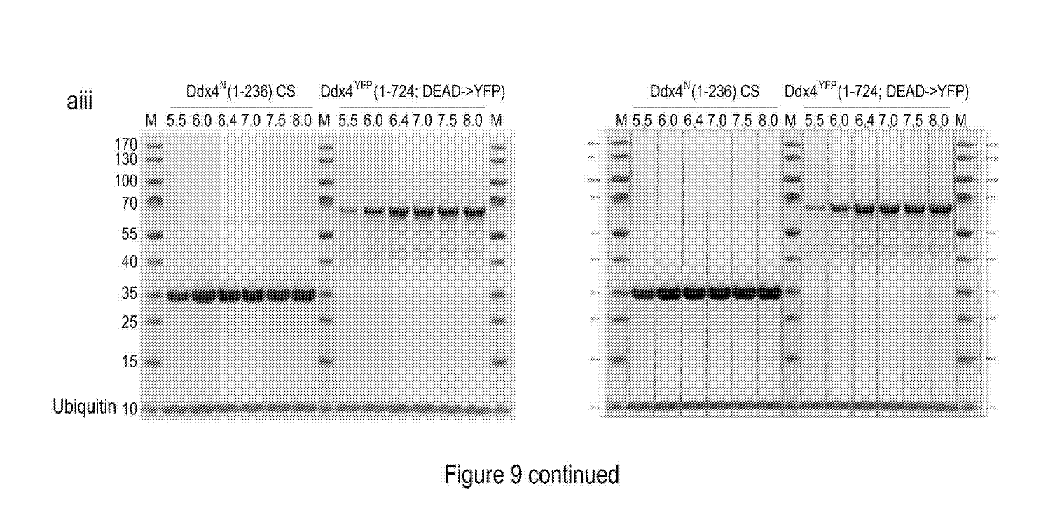

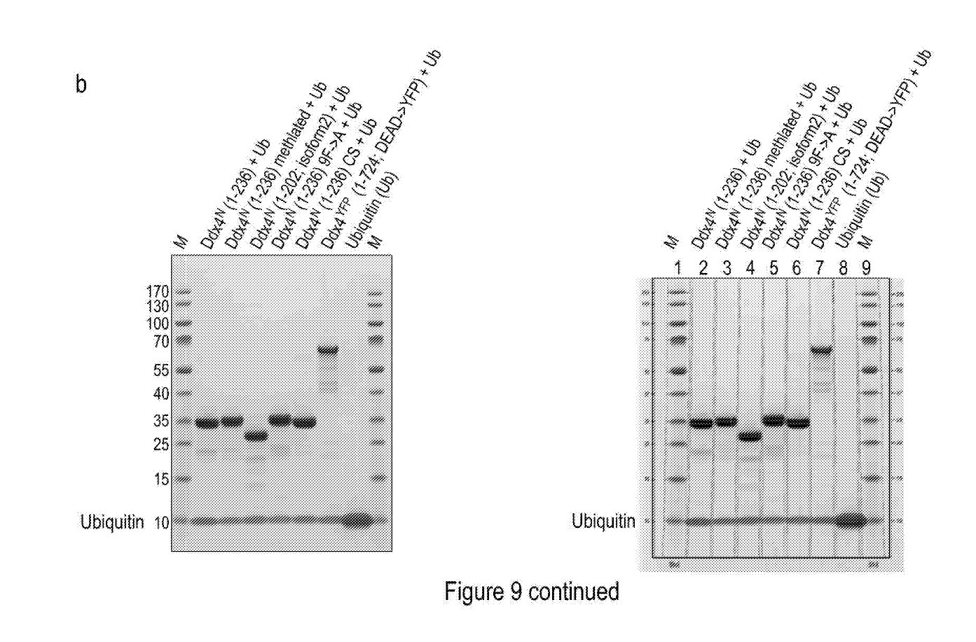

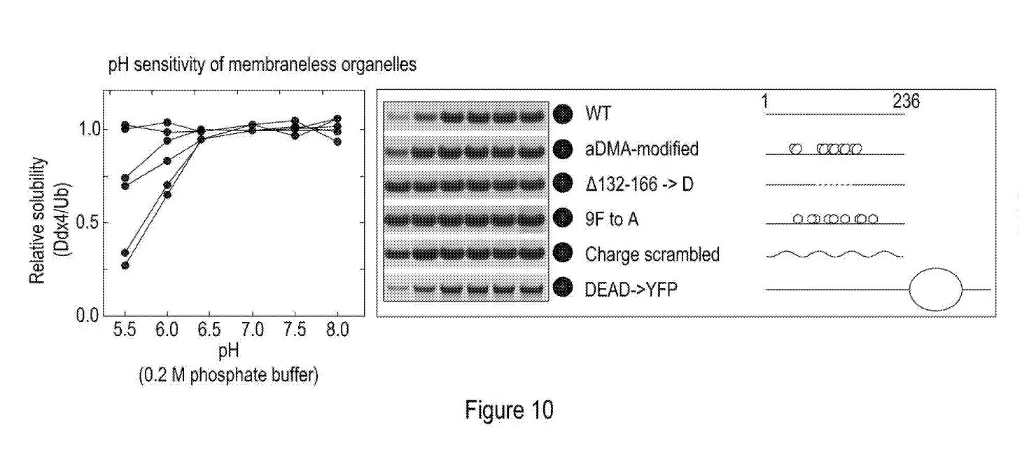

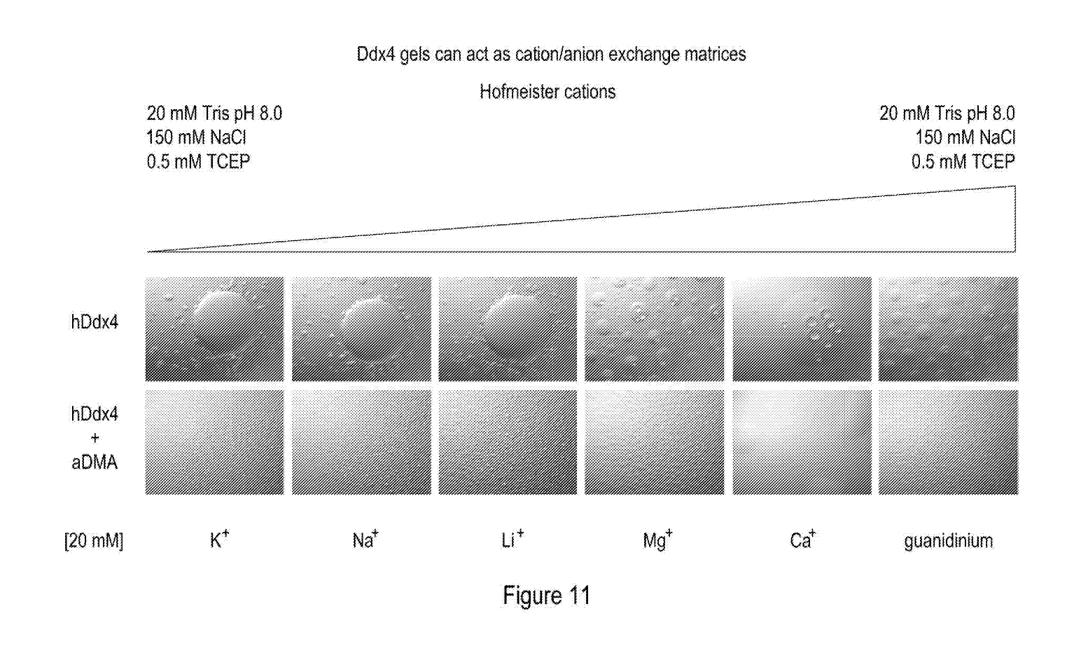

The skilled person will understand that the phase transition behaviour of any given self-assembling protein may be influenced by variation in one or more factors such as temperature, ionic strength, pH and protein concentration. Therefore, the phase transition of the self-assembling protein into the proteinaceous phase may be induced by a temperature between about 4° C. and about 50° C. The phase transition of the self-assembling protein into the proteinaceous phase may be induced by a temperature increase or decrease. The phase transition of the self-assembling protein into the proteinaceous phase may be induced by a pH increase or decrease. Additionally or alternatively, the ionic strength may be adjusted wherein the phase transition of the self-assembling protein into the proteinaceous phase may be induced by a salt (e.g. NaCl) concentration of between about 5 mM and about 5M. Additionally or alternatively, the ionic strength may be adjusted wherein the phase transition of the self-assembling protein into the proteinaceous phase may be induced by reducing a salt, such as NaCl, concentration. The ionic strength may be reduced by reducing NaCl concentration from about 300 mM to about 100 mM NaCl. Additionally or alternatively, the phase transition may be induced by a self-assembling protein concentration of between about 10 μM and about 1 mM. Description of the phase behaviour of a prototypical self-assembling protein is outlined in Nott et al., 2015, Molecular Cell 57, 936-947, which is herein incorporated by reference. Additionally or alternatively, the phase transition may be induced by pH conditions of between about pH2 and about pH10 (see FIGS. 9 and 10). Additionally or alternatively, the phase transition may be induced by the addition of molecular crowding agents such as dextrans/ficoll or PEGs, as described in Lin, Y., Protter, D. S., Rosen, M. K. & Parker, R. Mol Cell (2015) and Molliex, A. et al. Cell 163, 123-133 (2015). Combinations of all these phase transition induction factors may be applied to tune the phase behaviour of the self-assembling protein/proteinaceous phase. Hofmeister cations (for example selected from any one of K⁺, Na⁺, Li⁺, Mg²⁺, Ca²⁺, and guanidinium, or combinations thereof) and/or surfactants (such as PEG) can be provided and used to modulate the phase behaviour (see FIG. 11).

The self-assembling protein may comprise an intrinsically disordered protein, or fragment thereof, associated with membraneless organelles.

The self-assembling protein may comprise repeating 8-10 residue blocks of alternating net charge, and optionally an over-representation of FG, GF, RG, and GR motifs within the positively charged blocks. In this context, over-representation means ‘significantly more than would be expected by chance’ for example relative to an average human disordered protein sequence of the Uniprot database (www.uniprot.org/). The self-assembling protein may comprise FG and GF pairs spaced by 8-11 residues apart and RG and GR pairs to spaced 4 residues apart. The skilled person will be able to readily identify an appropriate self-assembling protein using the method of Nott et al., 2015, Molecular Cell 57, 936-947, which is herein incorporated by reference.

The self-assembling protein may comprise a protein derived from nuage, nuclear bodies, nuclear speckles, the spliceosome, the nucleolus, Cajal bodies, P-bodies or stress granules. In one embodiment, the self-assembling protein comprises Ddx4 protein. The self-assembling protein may comprise the disordered N-terminus of Ddx4 protein. The disordered N-terminus may comprise residue numbers 1-236 of the Ddx4 protein, or variants and/or truncations thereof. The DEAD-box helicase of Ddx4 may be replaced by a protein or peptide. For example, the DEAD-box helicase may be substituted for fluorescent marker protein such as GFP, RFP or YFP. In one embodiment the self-assembling protein does not comprise or consist of a fluorescence protein, such as GFP (green fluorescence protein). In another embodiment the self-assembling protein may comprise a fluorescence protein, such as GFP (green fluorescence protein), only in association (such as a fusion) with another self-assembling protein, such as Ddx4. For example, in examples where the DEAD-box helicase is substituted with a fluorescence protein.

In one embodiment the self-assembling protein does not comprise or consist of Ultrabithorax (Ubx).

In one embodiment the self-assembling protein does not directly bind to, or have affinity for, the target molecule. In one embodiment the self-assembling protein does not directly bind to, or have affinity for, the target molecule when the self-assembling protein is in its unassembled state. In another embodiment the self-assembling protein is not bound to the target molecule.

The self-assembling protein may comprise a human protein. In another embodiment, the self-assembling protein may comprise a non-human self-assembling protein. The non-human protein may be mammalian. In another embodiment, the non-human self-assembling protein may be bacterial. In another embodiment, the self-assembling protein may comprise a eukaryotic or prokaryotic protein.

The self-assembling protein may be selected from the group comprising Ddx4; Ddx3x; EWSR1; EIF4H; fragments/truncations and variants thereof; or combinations thereof.

The self-assembling protein may be selected from any one of the proteins listed in Table 1. The self-assembling protein may be a variant and/or truncation of a protein selected from any one of the proteins listed in Table 1. The proteins identified in Table 1 are human. However, equivalent non-human homologues may be provided for the self-assembling protein.

Variants of the self-assembling protein may include a protein having at least 70% sequence identity to any one protein identified in Table 1. In another embodiment, variants of the self-assembling protein may include a protein having at least 75% sequence identity to any one protein identified in Table 1. In another embodiment, variants of the self-assembling protein may include a protein having at least 80% sequence identity to any one protein identified in Table 1. In another embodiment, variants of the self-assembling protein may include a protein having at least 85% sequence identity to any one protein identified in Table 1. In another embodiment, variants of the self-assembling protein may include a protein having at least 90% sequence identity to any one protein identified in Table 1. In another embodiment, variants of the self-assembling protein may include a protein having at least 95% sequence identity to any one protein identified in Table 1. In another embodiment, variants of the self-assembling protein may include a protein having at least 98% sequence identity to any one protein identified in Table 1. In another embodiment, variants of the self-assembling protein may include a protein having at least 99% sequence identity to any one protein identified in Table 1. The sequence identity may be measured across the complete protein. In another embodiment, the sequence variation may be in regions of the protein outside of the repeating 8-10 residue blocks of alternating net charge, which optionally comprise an over-representation of FG, GF, RG, and GR motifs within the positively charged blocks.

Truncations of the self-assembling protein may include a protein having at least 20 amino acids of the proteins of Table 1, or variants or homologues thereof. Truncations of the self-assembling protein may include a protein having at least 30 amino acids of the proteins of Table 1, or variants or homologues thereof. Truncations of the self-assembling protein may include a protein having at least 40 amino acids of the proteins of Table 1, or variants or homologues thereof. Truncations of the self-assembling protein may include a protein having at least 50 amino acids of the proteins of Table 1, or variants or homologues thereof. Truncations of the self-assembling protein may include a protein having at least 60 amino acids of the proteins of Table 1, or variants or homologues thereof. Truncations of the self-assembling protein may include a protein having at least 80 amino acids of the proteins of Table 1, or variants or homologues thereof. Truncations of the self-assembling protein may include a protein having at least 100 amino acids of the proteins of Table 1, or variants or homologues thereof.

A variant of the self-assembling protein may alternatively comprise an elongated variant of the self-assembling protein.

In one embodiment, the self-assembling protein may comprise any one of EWSR1; LSM14A; NUCL; KHBRDS1; DDX4; DDX3X; RBM3; and EIF4H; or variants or homologues thereof; or combinations thereof. The above variants/truncations mentioned above for proteins of Table 1 may also equally apply to these self-assembling proteins.

TABLE 1
Human self-assembling proteins. Proteins are named using unique
UniProtKB AC/ID identifiers. Synonyms for which can be found using
http://www.uniprot.org/. The Uniprot IDs can be mapped against equivalent
accession/identification numbers in other databases using the tools in
http://www.uniprot.org/uploadlists/.

| | | | |
|---|---|---|---|
| ZC3H4 | EWS | RMXL3 | GAR1 |
| SRRM2 | LS14A | CHTOP | CO4A2 |
| MLL4 | CO7A1 | RS2 | WDR33 |
| FILA | TR150 | NUCL | MBD2 |
| RBP56 | BCLF1 | MTL14 | NU214 |
| AVEN | EIF3A | HNRPR | CORA1 |
| ILF2 | CO6A3 | PRC2A | RBM27 |
| UBAP2 | YLPM1 | CX018 | ZN469 |
| ZN579 | THOC4 | LS14B | EPIPL |
| ILF3 | PRC2C | F120A | SMD1 |
| HNRPK | SCAFB | RBM26 | RBM6 |
| CO6A2 | LENG8 | FGF2 | CO5A1 |
| COIA1 | KHDR1 | PRC2B | NUP98 |
| LARP1 | CB090 | SSF1 | HABP4 |
| ZHX1 | MRE11 | PERQ2 | CO9A1 |
| ROA3 | VIR | FBLL1 | HORN |
| CO6A1 | CCD61 | PR20A | CO6A5 |
| FA98A | CK096 | COIL | ZN326 |
| DDX4 | RBM33 | RIMB1 | SNUT1 |
| PRD10 | DDX3X | DDX42 | ZN318 |
| HNRL1 | CSTFT | MAGI3 | CX067 |
| CAPR1 | PABP2 | ZO2 | PNML2 |
| MCM8 | CNBP | NEUA | SRRM5 |
| MBD6 | DHX9 | MLL3 | COMA1 |
| ZO3 | GEMI7 | CCDC9 | DAZP1 |
| RBM3 | MLL2 | DPH1 | BCL7C |
| COKA1 | GCF | HDGR2 | BAZ2A |
| DACT3 | K2C1 | RBP2 | CDAN1 |
| DSRAD | ZC3H1 | DDX21 | MZF1 |
| AKAP8 | PRR12 | ZFP91 | CI004 |
| CA115 | COLQ | BAZ1A | AHDC1 |
| MYO15 | LSM4 | RAB44 | SRRM4 |
| RS10L | PROP | SRSF8 | BWR1B |
| SCAF8 | LSM11 | CECR6 | SPEG |
| CT75 | PKHA4 | K895L | TP53B |
| TUSC1 | RU17 | CELR3 | KHNYN |
| DNJB5 | COCA1 | MAGI1 | ESPL1 |
| SRR1L | LARP4 | SFPQ | IF4H |
| NOTC4 | MN1 | COGA1 | YI023 |

The self-assembling protein may be modified in order to control phase transition properties and/or target molecule absorption properties of the proteinaceous phase. In one embodiment, the self-assembling protein may be modified through the methylation of one or more arginine residues. The self-assembling protein may be modified through the methylation of 2 to 10 arginine residues. The self-assembling protein may be modified through the methylation of 5 to 6 arginine residues. In another embodiment, the self-assembling protein may be modified through the methylation of at least 2 arginine residues. In another embodiment, the self-assembling protein may be modified through the methylation of at least 4 arginine residues.

Advantageously methylation significantly destabilises the proteinaceous phase globules, lowering the transition temperature, for example by as much as 25° C. The extent of the destabilization of the droplets can be the equivalent of adding 100 mM of additional salt to the solution.

In another embodiment, the self-assembling protein may be modified through mutation or deletion of one or more amino acid residues of the self-assembling protein. The skilled person will understand that an appropriate modification may be provided depending on the phase transition behaviour required. For example, Nott et al. (2015, Molecular Cell 57, 936-947) describes substitution of residues 132-166 of Ddx4, where a single aspartate residue significantly modifies the phase transition behaviour.

Similarly, mutation of phenylalanine residues to alanine or modification of phenylalanine residues through fluorination may further modify the phase behaviour. Rearrangement of charged residues may modify the phase transition behaviour. Such mutations and modifications may be made in combination. In one embodiment, one or more charged residues may be modified. Additionally, or alternatively, one or more aromatic residues may be modified. Equivalent mutations may be made to any one of the proteins described in Table 1.

A plurality of proteinaceous phase globules may be provided. The plurality of proteinaceous phase globules may be uniform in composition and/or size. In another embodiment, different proteinaceous phase species may be provided in the same solution. For example, a mixture of two or more different proteinaceous phase species may be provided. The different proteinaceous phase species may each target different target molecules for absorption and/or different non-target molecules for exclusion. The different proteinaceous phase globules may be provided by the use of a different self-assembling protein species for each proteinaceous phase species.

The proteinaceous phase may be at least 500 nm in size as determined by the largest dimension. The plurality of proteinaceous phase globules may be at least 500 nm in size as an average of the population as determined by the largest dimension.

The plurality of proteinaceous phase globules may comprise two or more globules per ml of solution. The plurality of proteinaceous phase globules may comprise three, four, five, six, seven, eight, nine, or ten or more globules per ml of solution. The plurality of proteinaceous phase globules may comprise 100 or more globules per ml of solution. The plurality of proteinaceous phase globules may comprise 1000 or more globules per ml of solution. The plurality of proteinaceous phase globules may comprise 10000 or more globules per ml of solution. In one embodiment the total volume of the proteinaceous phase (including single or multiple proteinaceous globules) in the solution may be between about 0.5 μl to about 10 ml. In one embodiment the total content of the proteinaceous phase (including single or multiple proteinaceous globules) in the solution may be between about 0.001% to about 99% (v/v) of the solution.

In one embodiment, the proteinaceous phase may selectively exclude one or more, or all non-target molecules. In one embodiment, all non-target molecules may be excluded from absorption by the proteinaceous phase. In another embodiment, several different target molecule species may be absorbed into the proteinaceous phase, with at least one non-target molecule species excluded from absorption into the proteinaceous phase.

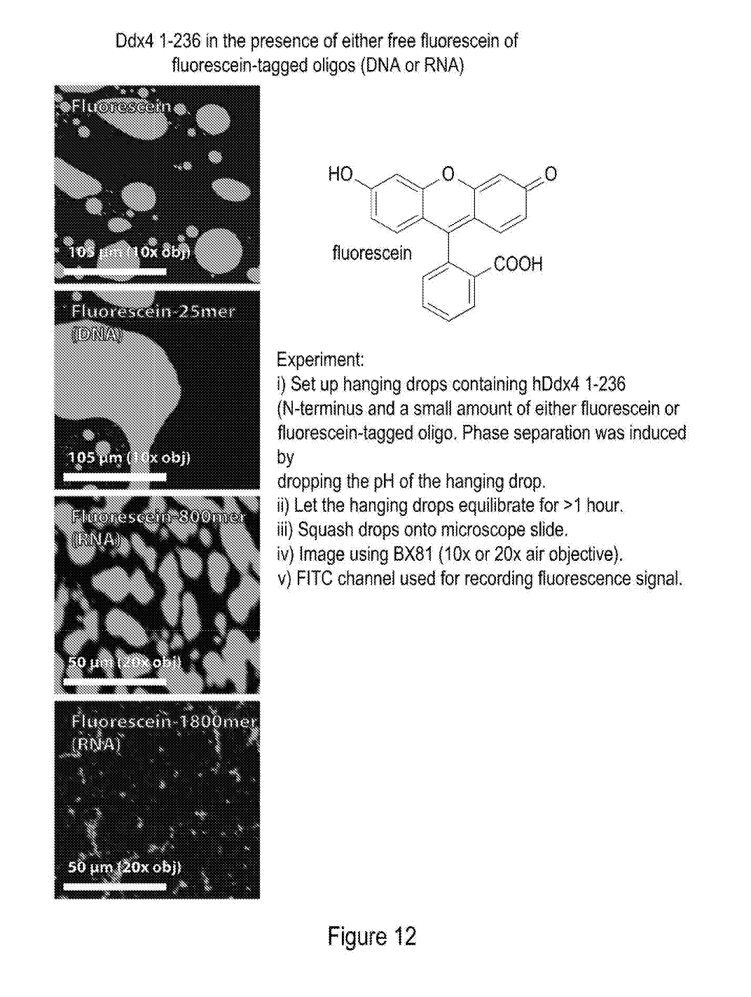

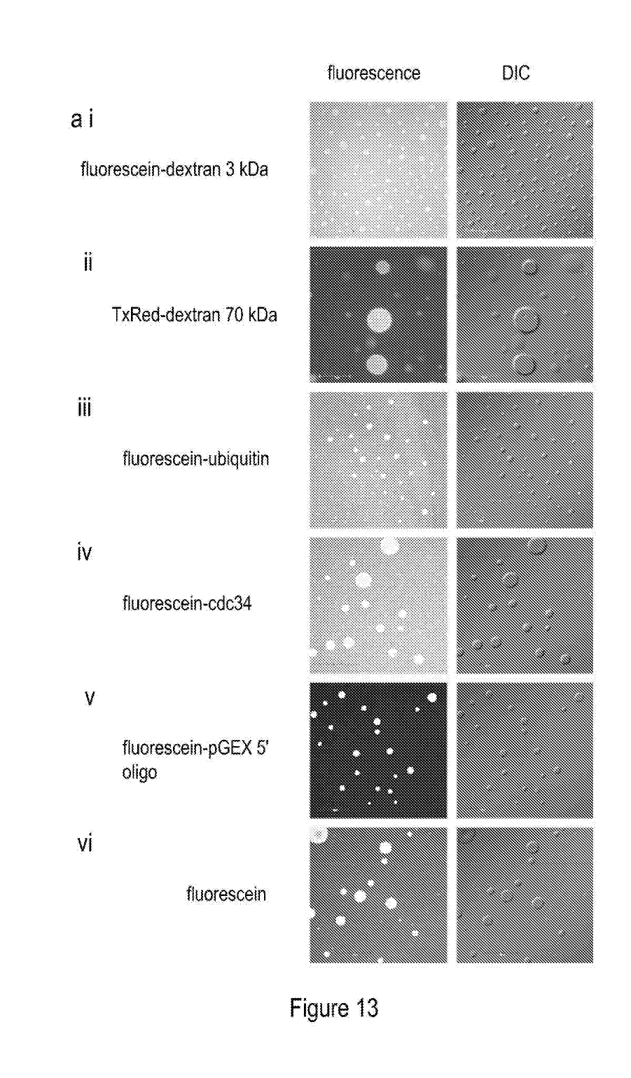

The target molecule may comprise a biomolecule. The target biomolecule may comprise a protein, peptide or nucleic acid, or analogues thereof. In another embodiment the target molecule may comprise any one of small molecules, natural or synthetic polymers, sugar chains, such as dextran, fatty acid chains; or combinations thereof. Small molecule target molecules may comprise a molecule with a MW of <900 Da. The target molecule may comprise a small molecule with one or more aromatic moieties, for example a poly-aromatic molecule. A small molecule with one or more aromatic moieties may comprise a fluorescein dye. See FIGS. 12 and 13.

The target nucleic acid may comprise an oligonucleotide. The target molecule may be single stranded nucleic acid. The target nucleic acid may comprise ssDNA. Additionally or alternatively, the molecule may comprise ssRNA. The target nucleic acid may comprise duplexes less than 20 base pairs. In another embodiment, the target nucleic acid may comprise RNA in structural conformation.

The target RNA may comprise total RNA (i.e. all the RNA of a cell). The target RNA may comprise one or more, or all of the RNA molecules selected from mRNA, polyA RNA, polysomal RNA, tRNA, ribosomal RNA, lincRNA, miRNA, piRNA, siRNA, SRP RNA, tmRNA, snRNA, snoRNA, SmY RNA, scaRNA, gRNA, aRNA, crRNA, tasiRNA, rasiRNA, land SK RNA.

The target nucleic acid may comprise nucleic acid with a secondary structure, such as hairpin structure. The target nucleic acid may comprise DNA and/or RNA hairpin molecules. The DNA and/or RNA hairpin molecules may have a stem length of between about 6 and about 20 nucleotides. The DNA and/or RNA hairpin molecules may have a stem length of between about 6 and about 15 nucleotides, alternatively between about 6 and about 10 nucleotides. For example, a nucleic acid with a secondary structure may be enriched by absorption into the proteinaceous phase more than an unstructured nucleic acid molecule. Structured nucleic acid may be identified by the skilled person, and may be aided by the use of tools such as Unafold (Markham, N. R. & Zuker, M. (2008) UNAFold: software for nucleic acid folding and hybridization. In Keith, J. M., editor, Bioinformatics, Volume II. Structure, Function and Applications, number 453 in Methods in Molecular Biology, chapter 1, pages 3-31. Humana Press, Totowa, N.J. ISBN 978-1-60327-428-9). Unafold can be used to indicate if a specific sequence is more likely to be structured.

The target nucleic acid may be 3 or more nucleotides in length. The target nucleic acid may be 5 or more nucleotides in length. The target nucleic acid may be between about 3 and about 20,000 nucleotides in length. The target nucleic acid may be between about 5 and about 20,000 nucleotides in length. The target nucleic acid may be between about 8 and about 20,000 nucleotides in length. See FIG. 12. In another embodiment, the target nucleic acid may be between about 8 and about 18,000 nucleotides in length. In another embodiment, the target nucleic acid may be between about 3 and about 15,000 nucleotides in length. In another embodiment, the target nucleic acid may be between about 5 and about 15,000 nucleotides in length. In another embodiment, the target nucleic acid may be between about 8 and about 15,000 nucleotides in length. In another embodiment, the target nucleic acid may be between about 3 and about 10,000 nucleotides in length. In another embodiment, the target nucleic acid may be between about 5 and about 10,000 nucleotides in length. In another embodiment, the target nucleic acid may be between about 8 and about 10,000 nucleotides in length. In another embodiment, the target nucleic acid may be between about 8 and about 5,000 nucleotides in length. In another embodiment, the target nucleic acid may be between about 8 and about 1,000 nucleotides in length. In another embodiment, the target nucleic acid may be between about 8 and about 500 nucleotides in length. In another embodiment, the target nucleic acid may be between about 8 and about 200 nucleotides in length. In another embodiment, the target nucleic acid may be between about 8 and about 100 nucleotides in length. In another embodiment, the target nucleic acid may be between about 8 and about 50 nucleotides in length. In another embodiment, the target nucleic acid may be between about 8 and about 25 nucleotides in length. In another embodiment, the target nucleic acid may be between about 8 and about 20 nucleotides in length. In another embodiment, the target nucleic acid may be between about 8 and about 18 nucleotides in length. In an embodiment wherein the nucleic acid is double stranded, the double stranded nucleic acid may be between about 8 and about 20 nucleotides in size, alternatively between about 8 and about 18 nucleotides in size.

Advantageously, it is found that the proteinaceous phase strongly absorbs nuclear RNAs containing an overrepresentation of CAXT motifs. Therefore, in one embodiment, the target nucleic acid may comprise nucleic acid comprising a CAXT motif, wherein X is A, T, C or G (and in the case of RNA T is U). In another embodiment, the target nucleic acid may comprise nucleic acid comprising an overrepresentation of CAXT motifs, wherein X is A, T, C or G (and in the case of RNA T is U). An overrepresentation of motifs or residues may be understood by the skilled person as more than, such as substantially more than, the average found in a population of nucleic acid molecules which are not target nucleic acid molecules.

In one embodiment, a target molecule, such as a nucleic acid, may be tagged with a nucleic acid molecule comprising one or more CAXT motifs (wherein X is A, T, C or G (and in the case of RNA T is U)) to enhance selective absorption of the target molecule. In another embodiment, a target nucleic acid molecule may be engineered to increase the number of CAXT motifs (wherein X is A, T, C or G (and in the case of RNA T is U)) to enhance selective absorption of the target molecule. In another embodiment, the target nucleic acid molecule may be a modified form of the nucleic acid molecule relative to wild type, wherein the modified form has been engineered to increase the number of CAXT motifs (wherein X is A, T, C or G (and in the case of RNA T is U)) relative to wild type.

The target peptide/protein may be between about 2 and about 300 amino acids in length. The target peptide/protein may comprise a multiple protein assembly, for example a virus particle or a multi-subunit protein. The target molecule, such as a peptide/protein including assemblies thereof, may have a molecular weight of between about 100 Da and about 10 MDa.

The target molecule may comprise a protein. In particular, the proteinaceous phase is capable of selectively excluding particular species of proteins. A target protein may have a partitioning Gibbs free energy (ΔGₚₐᵣₜ) of greater than 0 kJ mol⁻¹, resulting in net absorption. In another embodiment, the target protein may have a partitioning Gibbs free energy (ΔGₚₐᵣₜ) of greater than 1 kJ mol⁻¹ (i.e. enrichment inside the proteinaceous phase of greater than 1.5× compared to the bulk solution). In another embodiment, the target protein may have a partitioning Gibbs free energy (ΔGₚₐᵣₜ) of greater than 2 kJ mol⁻¹ (i.e. enrichment inside the proteinaceous phase of greater than 2.3× compared to the bulk solution). In another embodiment, the target protein may have a partitioning Gibbs free energy (ΔGₚₐᵣₜ) of greater than 3 kJ mol⁻¹ (i.e. enrichment inside the proteinaceous phase of greater than 3.4× compared to the bulk solution). In another embodiment, the target protein may have a partitioning Gibbs free energy (ΔGₚₐᵣₜ) of greater than 5 kJ mol⁻¹ (i.e. enrichment inside the proteinaceous phase of greater than 8× compared to the bulk solution). In another embodiment, the target protein may have a partitioning Gibbs free energy (ΔGₚₐᵣₜ) of greater than 8 kJ mol⁻¹ (i.e. enrichment inside the proteinaceous phase of greater than 28× compared to the bulk solution). In another embodiment, the target protein may have a partitioning Gibbs free energy (ΔGpart) of greater than 15 kJ mol⁻¹ (i.e. enrichment inside the proteinaceous phase of greater than 500× compared to the bulk solution). A concentration difference of the target molecular between inside and outside the proteinaceous phase may be converted into a partitioning Gibbs free energy (ΔGₚₐᵣₜ) using the equation:

ΔGₚₐᵣₜ=RT Ln(K)

where K is the ratio of target material inside to outside the proteinaceous phase.

In one embodiment, the target molecule is a molecule that is undesirable in the solution and it is targeted for removal, or temporary sequestration, from the solution. Additionally or alternatively, a desirable molecule in the solution may be selectively excluded from absorption into the proteinaceous phase as a non-target molecule.

In one embodiment, the target molecule may be a chaperone for separation/isolation of another molecule (herein termed the “molecule for separation”). For example fluorescein-tagged molecules may be chaperoned by the fluorescein, and absorbed into the proteinaceous phase (see the behaviour of unlabelled versus fluorescein-labelled ubiquitin in FIGS. 9, 10 and 13). The molecule for separation may be selectively excluded from the proteinaceous phase without the aid of the target molecule chaperone. In another embodiment, the molecule for separation may not be selectively absorbed into the proteinaceous phase without the aid of the target molecule chaperone.

The molecule for separation may be linked to the target molecule chaperone. The molecule for separation may be covalently bound to the target molecule chaperone. In another embodiment the bond may be ionic. Alternatively, the molecule for separation may be entangled with the target molecule chaperone. The molecule for separation may be tagged with the target molecule chaperone.

In one embodiment, the target molecule may be a chaperone for a biomolecule to be separated. For example, the target molecule may be a chaperone for a nucleic acid molecule to be separated. In another example, the target molecule may be a chaperone for a protein or peptide to be separated. In another example, the target molecule may be a chaperone for a small molecule to be separated. See FIGS. 12 and 13.

In an embodiment wherein the molecule for separation and the target molecule chaperone are proteins or peptides, they may be linked together by expression as fusion protein/peptide. In another embodiment, the molecule for separation and the target molecule chaperone may be covalently linked, for example using click-chemistry. In another embodiment, the molecule for separation and the target molecule chaperone may be linked by an affinity tag system such as avidin-biotin. In another embodiment, the molecule for separation and the target molecule chaperone may be linked by affinity, such as through the binding affinity of an antibody, antibody fragment, analogue or mimic thereof.

In an embodiment wherein the molecule for separation and the target molecule chaperone are nucleic acids, they may be linked together by base-pair complementary binding/hybridisation.

The molecule to be separated may be linked to the self-assembling protein. Therefore the self-assembling protein may itself act as a target molecule chaperone for the molecule to be separated. During phase transition the linked self-assembling protein may assemble into the proteinaceous phase, thereby absorbing the linked molecule for separation. In an embodiment wherein the molecule for separation and the self-assembling protein are linked together, they may be linked by expression as a fusion protein/peptide. For example, the molecule for separation may replace a DEAD-box helicase of the self-assembling protein. In another embodiment, the molecule for separation and the self-assembling protein may be covalently linked, for example using click-chemistry. In another embodiment, the molecule for separation and the self-assembling protein may be linked by an affinity tag system such as avidin-biotin. In another embodiment, the molecule for separation and the self-assembling protein may be linked by affinity, such as through the binding affinity of an antibody, antibody fragment, analogue or mimic thereof.

Advantageously, linking the self-assembling protein to the molecule to be separated can provide a tightly controlled pattern of absorption and dissolution by control of the phase transition. For example, the phase transition into a proteinaceous phase leads to substantially complete separation/absorption of the molecule to be separated into the proteinaceous phase, and this happens immediately upon the phase transition.

The heterogeneous mixture may comprise or consist of cell or tissue extract. The heterogeneous mixture may comprise or consist of biopsy material. The heterogeneous mixture may comprise or consist of whole cell extract. The heterogeneous mixture may comprise or consist of a mixture of nucleic acid species and/or sequences. The heterogeneous mixture may comprise a bodily fluid sample. In another embodiment, the heterogeneous mixture may comprise an environmental sample, such as water, air, or soil sample. The heterogeneous mixture may comprise a food or beverage sample.

The heterogeneous mixture may comprise a cell culture sample. The heterogeneous mixture may comprise pre-extracted nucleic acid. The heterogeneous mixture may consist of nucleic acid and a solute.

Where the heterogeneous mixture is a bodily fluid sample, it may be from a mammal. The mammal may be human. The heterogeneous mixture may comprise a blood or blood plasma sample. The heterogeneous mixture may be selected from any of the group comprising a sample of blood; blood plasma; mucous; urine; faeces; cerebrospinal fluid; tissue such as organ tissue, lung aspirate; or combinations thereof.

The non-target molecule may comprise any molecule in the heterogeneous mixture that is not of interest to be separated from the heterogeneous mixture. The non-target molecule may comprise a protein, peptide or nucleic acid, or analogues thereof. The non-target molecule may comprise any one of a small molecule (for example, molecules with a MW of <900 Da), natural or synthetic polymers, sugar chains, and fatty acid chains; or combinations thereof.

The non-target molecule may comprise double stranded nucleic acid molecules. For example, duplexes longer than 20 base pairs may be excluded by the proteinaceous phase. Therefore, the non-target molecule may comprise double stranded nucleic acid molecules of greater than 20 base pairs in length. In another embodiment, the non-target molecule may comprise double stranded nucleic acid molecules of greater than 19 base pairs in length. In another embodiment, the non-target molecule may comprise double stranded nucleic acid molecules of greater than 18 base pairs in length. In another embodiment, the non-target molecule may comprise double stranded nucleic acid molecules of greater than 17 base pairs in length. The double stranded nucleic acid molecule may comprise overhangs (i.e. may not be a perfect duplex). For example the double stranded nucleic acid molecule may comprise a restriction enzyme cut product, or a probe hybridised to a different length (e.g. longer) sequence.

The non-target molecule may be selected from the group comprising chromatin; HSP (heat shock protein), such oligomers of HSP16.5; αB-crystallin; wild-type GFP (green fluorescence protein); and fragments thereof; or combinations thereof. In one embodiment, the non-target molecule comprises chromatin. In one embodiment, the non-target molecule comprises HSP. In one embodiment, the non-target molecule comprises oligomers of HSP16.5. In one embodiment, the non-target molecule comprises αB-crystallin. In one embodiment, the non-target molecule comprises wild-type GFP.

The non-target molecule may comprise a protein. In particular, the proteinaceous phase is capable of selectively excluding particular species of proteins. A non-target protein may have a partition Gibbs free energy (ΔGₚₐᵣₜ) of less than 0 kJ mol⁻¹, representing net exclusion from the proteinaceous phase. In another embodiment, the non-target protein may have a partition Gibbs free energy (ΔGₚₐᵣₜ) of less than −1 kJ mol⁻¹ (i.e. exclusion from the proteinaceous phase by more than a factor of 1.5× compared to the bulk solution). In another embodiment, the non-target protein may have a partition Gibbs free energy (ΔGₚₐᵣₜ) of less than −2 kJ mol⁻¹ (i.e. exclusion from the proteinaceous phase by more than a factor of 2.2× compared to the bulk solution). In another embodiment, the non-target protein may have a partition Gibbs free energy (ΔGₚₐᵣₜ) of less than −5 kJ mol⁻¹ (i.e. exclusion from the proteinaceous phase by more than a factor of 8× compared to the bulk solution). In another embodiment, the non-target protein may have a partition Gibbs free energy (ΔGₚₐᵣₜ) of less than −7 kJ mol⁻¹ (i.e. exclusion from the proteinaceous phase by more than a factor of 18× compared to the bulk solution). A concentration difference of the target molecular between inside and outside the proteinaceous phase may be converted into a partitioning Gibbs free energy (ΔGₚₐᵣₜ) using the equation:

ΔGₚₐᵣₜ=RT Ln(K)

Where K is the ratio of target material inside to outside the proteinaceous phase.

In one embodiment a molecule to be excluded may be linked to a non-target molecule. For example, the non-target molecule may act as a chaperone for a linked molecule, which selectively prevents absorption into the organelle by exclusion of the non-target molecule chaperone.

In an embodiment wherein the molecule for exclusion and the non-target molecule chaperone are proteins or peptides, they may be linked together by expression as fusion protein/peptide. In another embodiment, the molecule for exclusion and the non-target molecule chaperone may be covalently linked, for example using click-chemistry. In another embodiment, the molecule for exclusion and the non-target molecule chaperone may be linked by an affinity tag system such as avidin-biotin. In another embodiment, the molecule for exclusion and the non-target molecule chaperone may be linked by affinity, such as through the binding affinity of an antibody, antibody fragment, analogue or mimic thereof.

In an embodiment wherein the molecule for exclusion and the non-target molecule chaperone are nucleic acids, they may be linked together by base-pair complementary binding/hybridisation.

Target and/or non-target molecules may be labelled to aid their localisation and identification. The skilled person will be familiar with appropriate labels such as dyes, fluorescent labels, microparticles, radioactive labels, probes, and the like.

According to another aspect of the invention, there is provided a method of selective separation of target molecules from a heterogeneous mixture of target molecules and non-target molecules in a solution, the method comprising:
tagging the target molecule with a self-assembling protein capable of assembling into a proteinaceous phase, wherein the proteinaceous phase comprises a matrix of the self-assembling proteins;
inducing the assembly of the self-assembling protein into the proteinaceous phase,
wherein the tagged target molecules are internalised into the matrix of the proteinaceous phase during the assembly thereby selectively isolating the target molecules from the non-target molecules in the solution.

Tagging the target molecule with a self-assembling protein may comprise linking the target molecule with a self-assembling protein as described herein. For example, the linking may comprise a covalent linkage or affinity tagging.

Following absorption of the target molecule, the methods of the invention may further comprise the step of separating/isolating the proteinaceous phase from the solution. The separation may comprise sedimentation by centrifugation (for example between 10 g and 23,000 g) of the proteinaceous phase and decanting the remaining solution away from the sediment. The sedimentation may form a larger coalesced proteinaceous phase/dense liquid phase at the bottom of the container. The proteinaceous phase may be washed one or more times with a wash or re-suspension solution. The proteinaceous phase may be resuspended in a resuspension solution to form an isolated proteinaceous phase suspension.

Alternatively or additionally, the separation may comprise filtration or capture of the proteinaceous phase, for example in a matrix, mesh or column.

Additionally or alternatively, the proteinaceous phase may be tagged with an affinity tag or magnetic tag to aid separation. The tag may comprise a GST-tag or 6His tag, or the like.

Advantageously, the affinity purification tags do not need to be cleaved from the organelle-forming protein for phase separation to be easily controllable.

In another embodiment, the proteinaceous phase may be tagged with a molecule that causes it to float to the surface in a solution, thereby facilitating isolation of the proteinaceous phase by skimming or decanting it off the surface of the solution.

The phase transition of the proteinaceous phase may be reversed in order to dissolve the proteinaceous phase, thereby releasing the target molecule. The phase transition of the proteinaceous phase may be reversed for example, by a change in temperature, and/or pH, and/or ionic strength.

In one embodiment the separation of the target molecule from the heterogeneous mixture in the solution is a temporary separation. In particular, the phase transition of the proteinaceous phase may be reversed in order to dissolve the proteinaceous phase, thereby releasing the target molecule back into the solution. The reversal of the phase transition may be induced by a change in temperature, pH and/or salt concentration. The reversal of the phase transition may be after a desired reaction has occurred in the solution.

According to another aspect of the invention, there is provided a method of Ion Torrent Sequencing of RNA, wherein RNA extraction is provided by isolating the RNA from a solution according to the method of the invention herein.

The skilled person will be familiar with conventional methods of Ion Torrent Sequencing of RNA. It is understood that the RNA extraction steps of such methods can be readily substituted with the method of RNA isolation described herein.

According to another aspect of the invention, there is provided a composition comprising the self-assembling proteins as described herein, wherein the self-assembling proteins are capable of assembling into a matrix to form a proteinaceous phase.

The self-assembling proteins of the composition may be in a reversible amorphous solid (glassy) state. The composition may consist essentially of the self-assembling proteins as described herein. In another embodiment, the self-assembling proteins of the composition may be in solution. The solution of the composition may be a carrier comprising water. The solution of the composition may be a buffer solution. The solution of the composition may not comprise cell extract. The self-assembling proteins may be capable of assembling into a matrix in solution to form a proteinaceous phase.

The self-assembling protein(s) may be isolated, for example, isolated from other cell constituents. The self-assembling protein may be a recombinant protein.

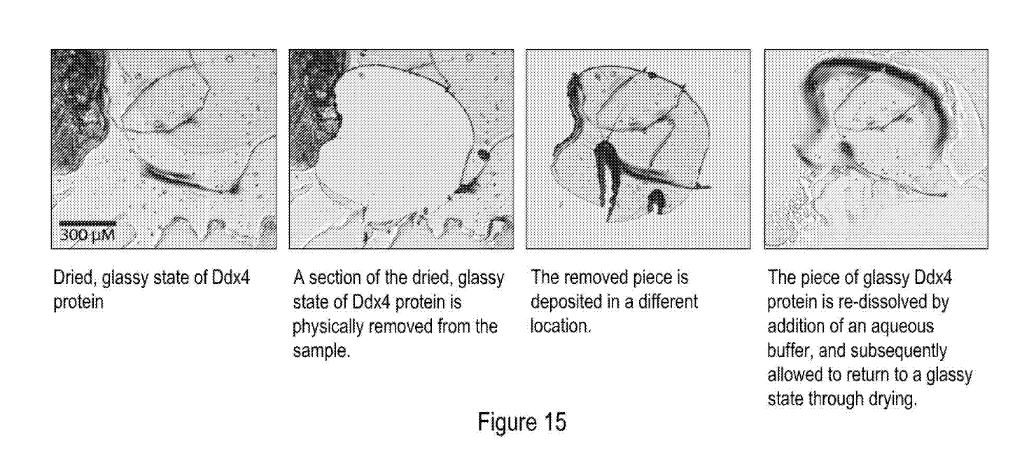

The composition may comprise a carrier. The carrier may comprise a buffer. The composition may be in the form of a solution, lyophilised powder, or as a dried amorphous solid (glass). The dried, glassy form of the proteinaceous phase may be described as an amorphous solid. A demonstration of the physical properties of the glassy-state of the proteinaceous phase is shown in FIG. 15.

According to another aspect of the invention, there is provided a kit for selective separation and/or modification of target molecules from a heterogeneous mixture of the target molecules and non-target molecules in a solution, wherein the kit comprises:
the self-assembling proteins described herein, which are capable of assembling into a matrix in solution to form a proteinaceous phase.

The kit may further comprise a target molecule chaperone for tagging/linking to a molecule of interest.

The self-assembling proteins of the kit may be in a reversible amorphous solid (glassy) state. The composition may consist essentially of the self-assembling proteins as described herein. The solution for reconstitution of the self-assembling proteins may be provided separately. In another embodiment, the self-assembling proteins of the kit may be in solution. The solution of the kit may be a carrier comprising water. The solution of the composition may be a buffer solution. The solution of the kit may not comprise cell extract. The self-assembling proteins of the kit may be capable of assembling into a matrix in solution to form a proteinaceous phase.

According to another aspect of the invention, there is provided use of self-assembling proteins described to selectively isolate target molecules from a heterogeneous mixture of the target molecules and non-target molecules in a solution.

The use may be for a diagnostic assay. The use may be for environmental sampling.

The self-assembling proteins may be capable of phase transition to form a proteinaceous phase.

Methods and compositions of the invention herein may be used for denaturing dsDNA, for example to open up a replication fork in DNA, or to open up regions of DNA to be hyper accessible to DNA modification enzymes (such as DNA repair enzymes).

In another embodiment, methods and compositions of the invention herein may be used for stabilising ssRNA, for example to purify regulatory RNA molecules from disordered RNA molecules, or for protecting RNA molecules from degrading enzymes.

The methods herein may be in vitro.

In one embodiment, the proteinaceous phase may be provided within a cell (or population of cells), for example to purify or isolate target molecules within a cell (or population of cells) prior to lysis. In one embodiment, the proteinaceous phase may be provided within a cell for sequestering one or more target molecules in the cell from other cell constituents. The sequestration may be temporary/reversible.

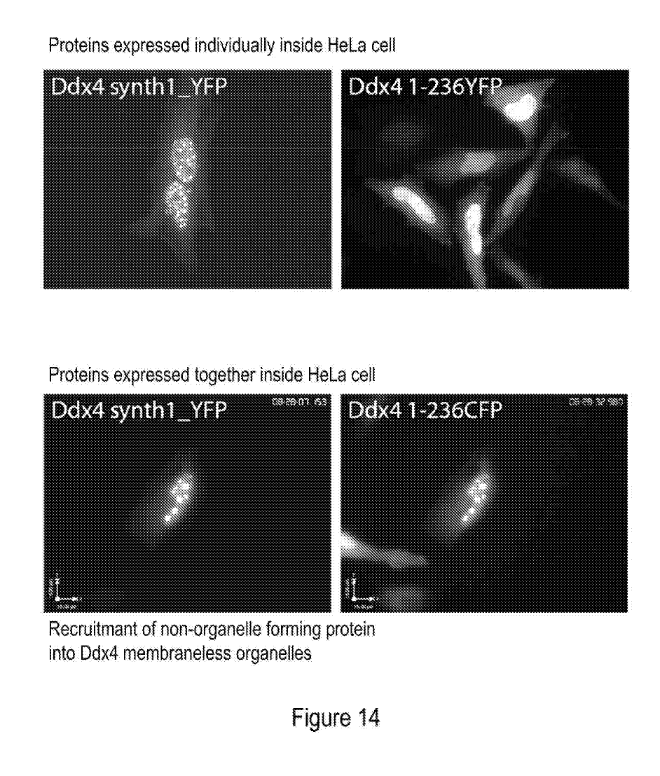

The proteinaceous phase may be provided within a cell (or population of cells) by expressing the self-assembling protein in the cell. The self-assembling protein may be endogenous and the over-expression of this protein is induced by genetic modification of the cell (or population of cells). For example additionally copies of the gene encoding the endogenous self-assembling protein may be transfected into the cell (or population of cells). Additionally or alternatively, the endogenous gene may be provided with a stronger and/or inducible promoter. In another embodiment, the cell (or population of cells) may be genetically modified with recombinant nucleic acid encoding an exogenous self-assembling protein. In one embodiment, the self-assembling protein may be expressed, or overexpressed, together with a target molecule. The self-assembling protein and target molecule may be expressed as a fusion protein. A demonstration of the sequestration of a target protein within proteinaceous globules formed by an organelle-forming protein is shown in FIG. 14.

Where reference is made to a protein sequence, the skilled person will understand that one or more substitutions may be tolerated, optionally two substitutions may be tolerated in the sequence, such that it maintains its function. References to sequence identity may be determined by BLAST sequence alignment (www.ncbi.nlm.nih.gov/BLAST/) using standard/default parameters. For example, the sequence may have 99% identity and still function according to the invention. In other embodiments, the sequence may have 98% identity and still function according to the invention. In another embodiment, the sequence may have 95% identity and still function according to the invention. In another embodiment, the sequence may have 90%, 85%, or 80% identity and still function according to the invention.

The skilled person will understand that optional features of one embodiment or aspect of the invention may be applicable, where appropriate, to other embodiments or aspects of the invention.

There now follows by way of example only a detailed description of the present invention with reference to the accompanying drawings, in which;

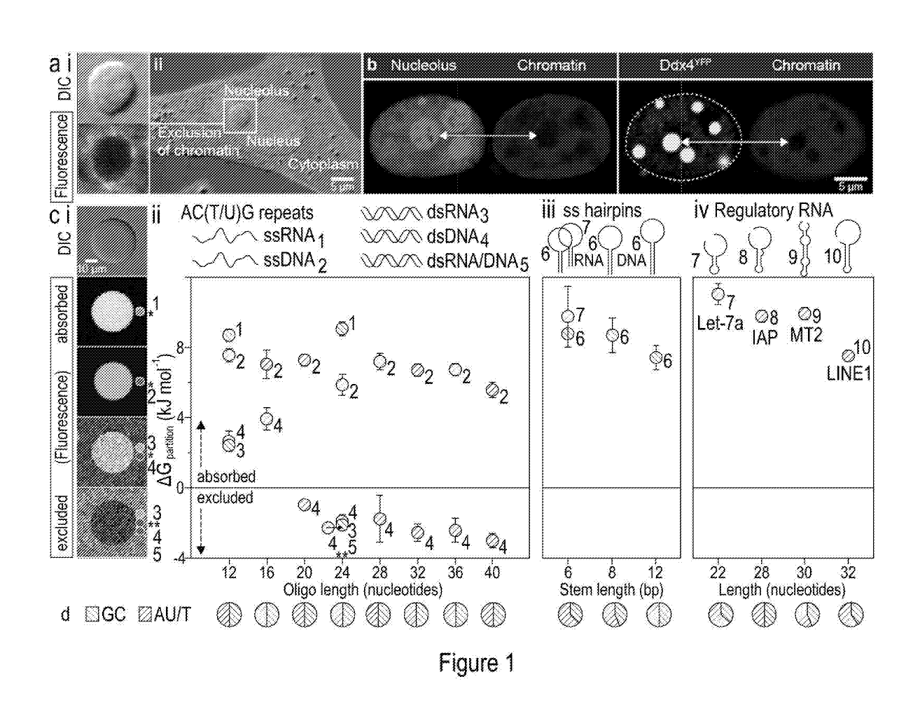

FIG. 1|Membraneless organelles selectively partition oligonucleotides. ai, Nucleoli of cultured cells are visible as dense, spherical droplets and exclude fluorescently-stained chromatin. (ii, Nucleolus from i shown in the context of the whole cell. b, Nucleoli, Ddx4YFP organelles⁶ and chromatin visualised together in a HeLa cell nucleus. Both Nucleoli and Ddx4YFP organelles exclude chromatin. c, Ddx4^{N1} organelles differentially partition nucleic acids in vitro. i and ii, Partitioning of a series of DNA and RNA oligonucleotides made of ACTG of ACUG repeats. Duplexes longer than 20 base pairs are predominantly excluded from Ddx4 organelles, whereas shorter duplexes and single-stranded oligonucleotides are absorbed. Coloured circles and asterisks in i correspond to the nucleic acids in ii. DNA and RNA hairpins with either synthetic (iii) or physiological (iv) sequences are absorbed into Ddx4^{N1} organelles. d, Nucleotide composition of sequences used in this study (table 3).

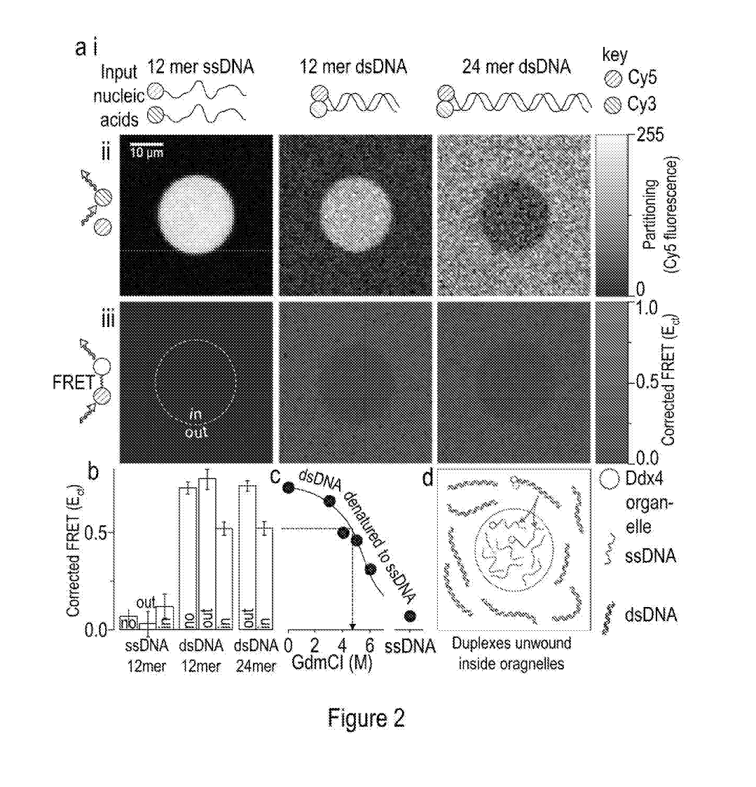

FIG. 2|Ddx4N1 organelles destabilise oligonucleotide duplexes. a, 12mer ssDNA and 12mer and 24mer dsDNA tagged with Cy3 (donor) and Cy5 (acceptor) fluorophores (i) were used to simultaneously measure nucleic acid partitioning (ii) and FRET (iii) inside and outside Ddx4N1 organelles (section S.11). b, Quantitation of the corrected FRET signals from inside and outside Ddx4N1 organelles. c, Chemical denaturation of the 12mer FRET-pair dsDNA by guanidinium chloride determined using a fluorimeter assay (section S.12). d, Schematic depiction of the dissociation of nucleic acid duplexes upon entering Ddx4N1 organelles.

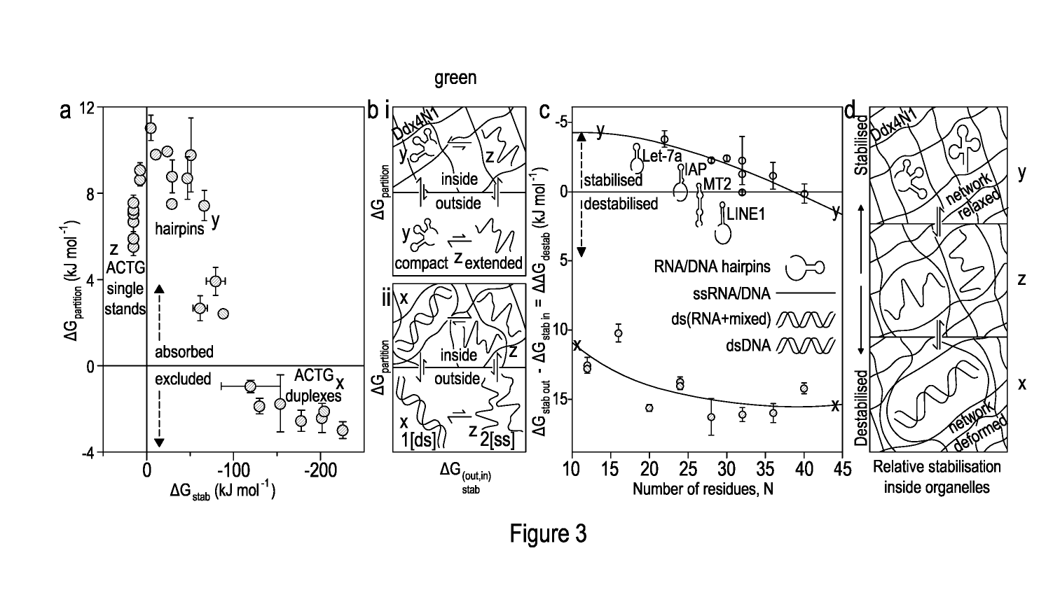

FIG. 3|Ddx4N1 organelle partitioning depends on the stability of the nucleic acid structure. a, Ddx4N1 organelle partitioning for all the sequences shown in FIG. 1c as a function of stability. Colour scheme as in FIG. 1c. b, The partitioning data can be interpreted using equilibrium schemes. In the case of single-stranded oligonucleotides, the stabilisation corresponds to folding (i), and in the case of double-stranded oligonucleotides, the stabilisation corresponds to melting (ii) c, Using the measurement of the partition free energy of the double-stranded DNA and the corresponding single-stranded DNAs together with the total concentration and the stability outside of the organelle, it is possible to calculate the stability inside the organelle, and hence the change in stability (red (X), bii). In a similar way, the change in stability for the structured RNA and DNA hairpins can be estimated (green (Y), bi). d, The data suggest that both extended and rigid nucleic acid duplexes are destabilised in the organelle interior, favouring instead compact, single chains that will minimally distort the interior of the underlying droplets. (Key: Red—X; Green—Y; Blue—Z).

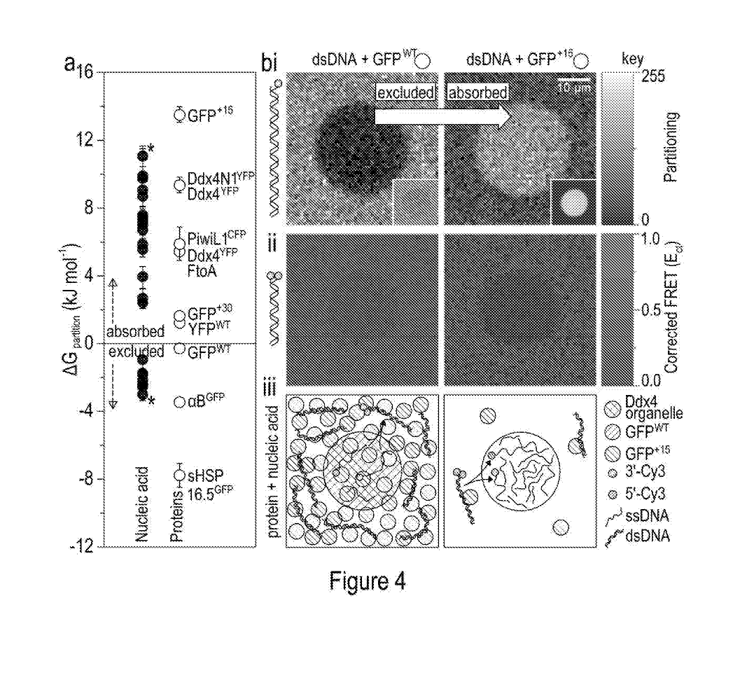

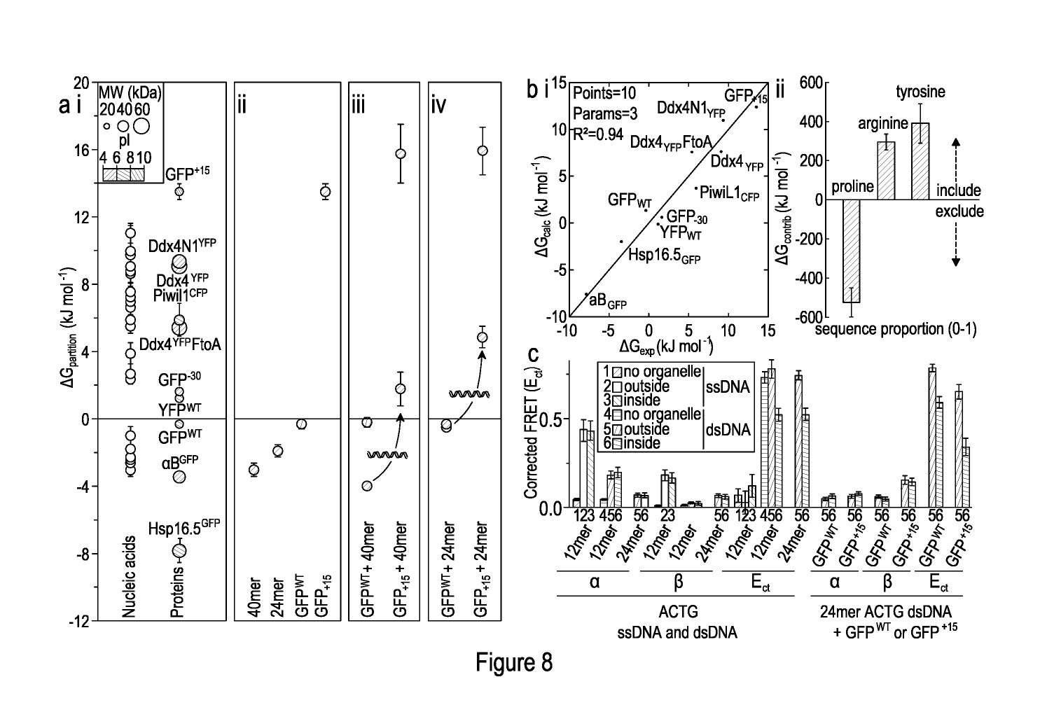

FIG. 4|Ddx4N1 organelles differentially partition proteins, which in turn can act as chaperones to import dsDNA. a, Proteins (white-filled circles) show a wider range of partitioning than nucleic acids (grey/*). The data can be quantitatively explained from sequence information alone (FIG. 8 bi, ii). b i, 40mer dsDNA is excluded from Ddx4N1 organelles in the presence of GFP^{WT}, but absorbed in the presence of GFP⁺¹⁵. ii, FRET images of 24mer dsDNA in the presence of GFP^{WT} and GFP⁺¹⁵. In both cases, FRET is reduced inside the Ddx4N1 organelle. iii, Schematic depiction showing the exclusion of long nucleic acid duplexes in the presence of GFP^{WT}, and the absorption and dissociation to single strands of long duplexes in the presence of GFP+15.

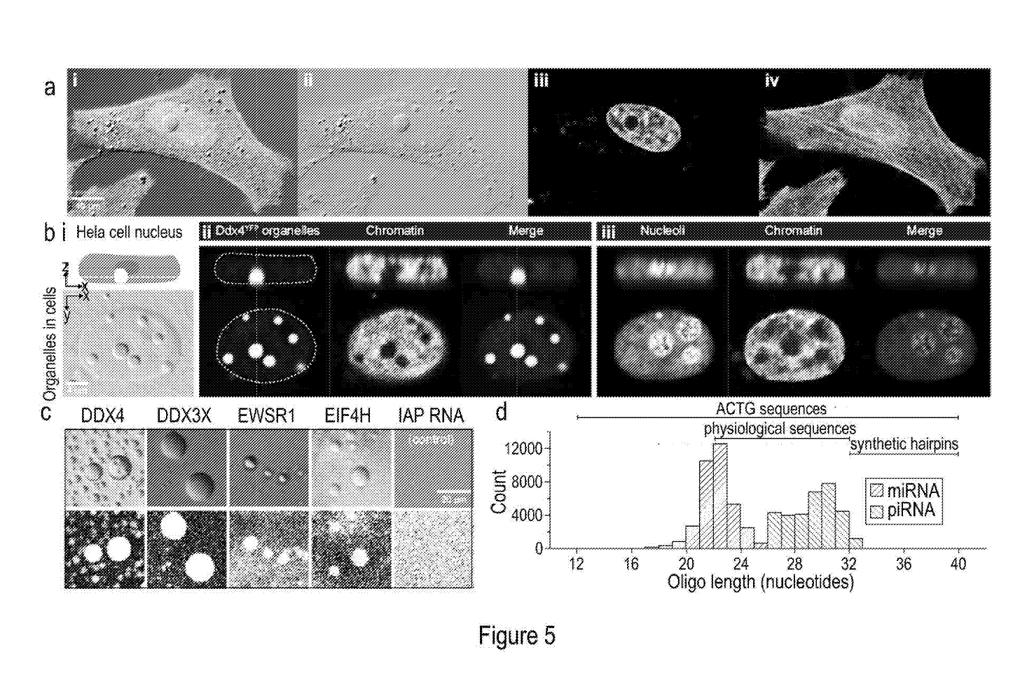

FIG. 5|a, Chromatin is excluded by the nucleolus and Ddx4N1 organelles. Fluorescence and differential interference contrast (DIC) overlay (i), DIC (ii), chromatin stained with Hoechst dye (iii) and Ddx4YFP_FtoA (soluble variant of Ddx4N1 dispersed in the cytoplasm and nucleus for additional contrast, iv). b, HeLa cell nucleus containing Ddx4YFP organelles, counter stained to visualise chromatin and nucleoli. i, Schematic representation of the xz view through the HeLa cell nucleus, showing Ddx4YFP organelles (yellow) and nucleoli (magenta). Dashed lines indicate, for the respective organelles, the z-slice (i.e. xy plane) shown in ii and iii. Both Ddx4YFP organelles and nucleoli exclude bulk chromatin in the nucleus (ii and iii). c, DIC and confocal fluorescence images showing the partitioning of 28mer IAP RNA in organelles formed from a range of membraneless organelle-forming proteins in vitro. d, Distribution of the lengths of all miRNAs and all human piRNAs in the mirBASE and piRNAbank databases, respectively, compared to those used in this study (labelled horizontal bars). miRNA and piRNA are classes of small interfering RNAs involved in translational repression, mRNA degradation, transposon suppression and epigenetic regulation.

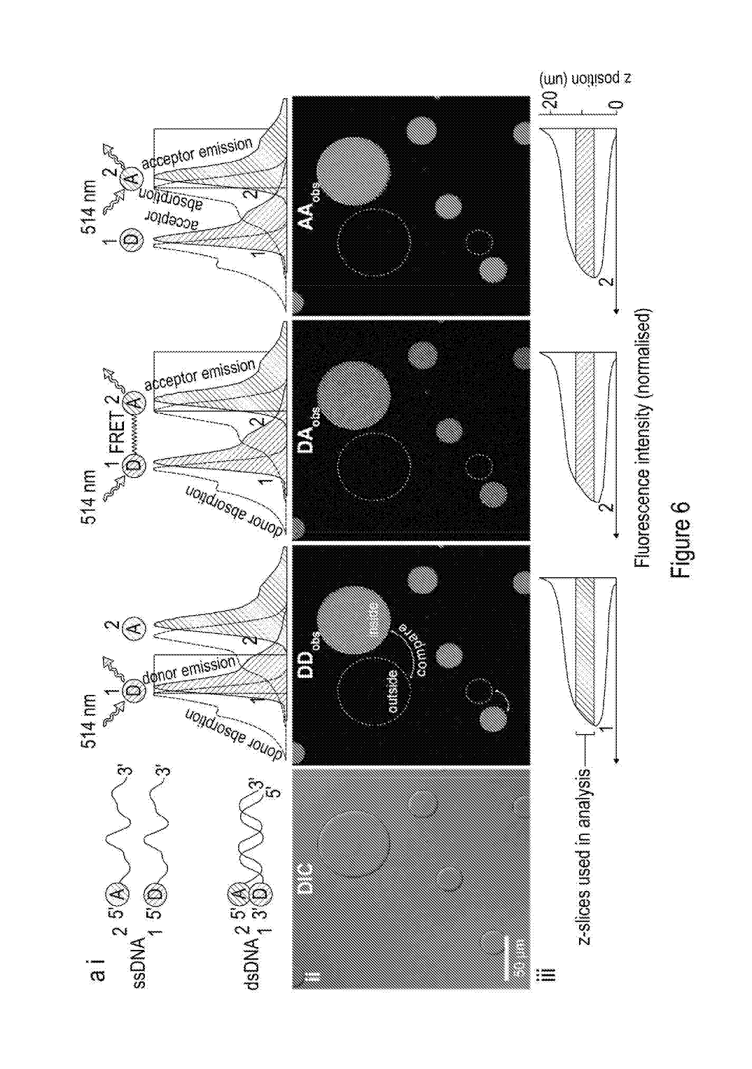

FIG. 6|ai, Schematic depiction of fluorescently labelled single and double-stranded ACTG DNAs used in FRET experiments. Cy3 (D) and Cy5 (A) absorption spectra are shown as dashed lines, and emission spectra are coloured yellow/marked “1” (Cy3) and orange/marked “2” (Cy5). Cy3 was excited at 514 nm (543 nm in experiments also containing GFPs; FIG. 4b and FIG. 8aii-iv) and Cy5 was excited at 633 nm. Grey rectangles indicate the wavelengths over which fluorescence emission was typically collected. ii, single xy slices extracted at equivalent positions from the fluorescence z-stacks recorded during a representative FRET experiment. iii, z-axis profiles of the fluorescence image stacks. Coloured bars indicate the regions of the image stacks used in the analysis.

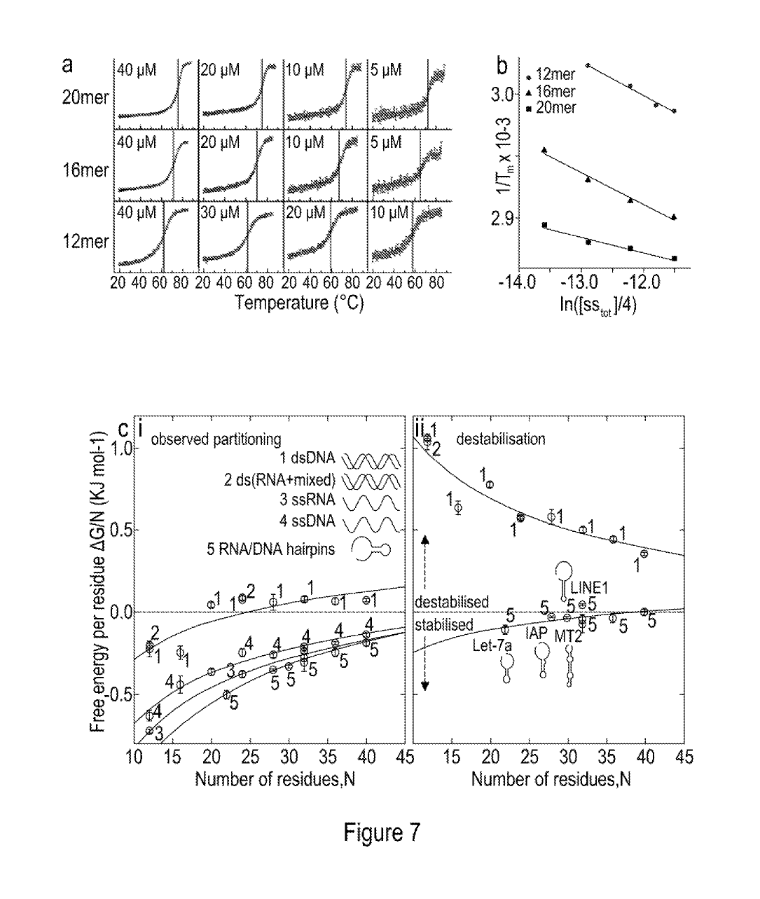

FIG. 7|a, Annealing profiles of the 12mer, 16mer and 20mer ACTG dsDNA. Vertical bars show the midpoint of the curve, at which the sample contains 50% duplex. b, The points and total concentration can be used together to get an experimental estimate of ΔG_{stab} (section S.7). c, The free energy per residue is observed to decrease with length, with a scaling consistent with a model of topological frustration (solid lines, section S.11). Both double and single stranded DNA, and hairpin and unstructured RNA/DNA were found to have very different partition free energies (i). This can be explained by destabilisation of the structure inside the organelles (ii). The destabilisation free energy per residue is shown. The free energy per individual base pair for each of the processes is reduced with length, consistent with topological frustration inside the liquid drops4.

FIG. 8|a, Ddx4 organelles differentially partition proteins, which in turn can act as chaperones to import and unwind dsDNA. ai, partitioning of proteins and nucleic acids. ii-iv, GFP⁺¹⁵ can chaperone otherwise excluded duplexes inside Ddx4 organelles. bi, Correlation between experimentally determined partition free energies, and those calculated through a sequence analysis. Including only three properties of the protein sequence is sufficient to calculate the partition free energy of the dataset with an R² of 0.94. ii, The optimised coefficients required to obtain the correlation shown (table 4). Knowledge of the proportion of proline, arginine and tyrosine in the protein sequence is sufficient to predict the protein partition free energy. c, Summary of all FRET data in the study, including correction factors and corrected FRET_{(Ect)} values.

FIG. 9|Experiment to test the pH-dependence of the phase transitions of Ddx4 protein, mutants of Ddx4, and a post translationally modified version. The proteins in this experiment are listed in table 10. In each case, the Ddx4 variant was fully dissolved in solution, and mixed with ubiquitin as an internal control. The two-protein mixtures were introduced to buffers covering a range of pH spanning pH 5.5 to 8.0. Samples at different pHs were incubated on ice for 30 minutes and spun in a benchtop centrifuge at 10000 g for 1 min to sediment the proteinaceous phase, if present. Following a repeat of the incubation on ice for 30 minutes and centrifugation steps, the supernatant of the samples was aspirated and its contents resolved using sodium dodecyl sulphate polyacrylamide gel electrophoresis (SDS-PAGE). Densitometry was performed on coomassie-stained SDS-PAGE gels to quantitate the pH-sensitivity of the Ddx4 proteins with respect to ubiquitin, which was not affected by either pH or the presence of the proteinaceous phase, if present.

FIG. 10|Quantitation of the pH-sensitivity of Ddx4 organelle-forming protein and Ddx4 protein variants to changes in solution pH. At pHs above 6.5, all Ddx4 proteins were soluble and did not undergo phase separation to form a proteinaceous (organelle) phase. The solubility of the 9FtoA and Δ132-136→D constructs was unaffected by changes in solution pH between pH 5 and 8. WT and DEAD→YFP proteins were induced to undergo phase separation below pH 6.5, forming droplets of a dense proteinaceous phase that was sedimented by centrifugation. As a result, these proteins were depleted from the bulk aqueous solution. aDMA-modified and charged-scrambled Ddx4 proteins underwent similar phase separation below pH 6.5, but were depleted from the bulk aqueous solution to a lesser extent.

FIG. 11|Induction and properties of the Ddx4 proteinaceous phase and can be modified and tuned by the addition of various charged ions into the solution. In this experiment, a range of cations from the Hofmeister series were included in the solution, and phase separation observed after 24 hours. A range of morphologies is seen that changes as the ion additive changes from kosmotropic to chaotropic.

FIG. 12|Ddx4 proteinaceous phase strongly absorbs free fluorescein dye, and single-stranded nucleic acids (DNA and RNA) conjugated to fluorescein dye. The nucleic acids tested ranged in length from 25 to 1800 nucleotides.

FIG. 13|Ddx4 organelles selectively absorb fluorescently-tagged polysaccharides (dextrans), proteins (cdc-34 and ubiquitin) and nucleic acids (pGEX 5′ oligo). 3 kDa dextran and 70 kDa dextran, labelled with fluorescein and TexasRed, respectively, are both absorbed into the membraneless organelle interior. Fluorescein-ubiquitin and fluorescein-cdc34 are similarly absorbed into the organelle interior. Free fluorescein dye and a 23mer DNA oligo labelled with fluorescein are similarly absorbed into the organelle interior. This experiment demonstrates that a small molecule such as fluorescein can be used as an importer for a molecule that did not otherwise partition (ubiquitin, FIGS. 9 and 10).

FIG. 14|Ddx4 organelles can localise non-organelle-forming proteins in cells. Ddx4 synth1_YFP corresponds to the Ddx4 protein in which the DEAD-box helicase domain is replaced with YFP. Ddx4 synth1_YFP forms membraneless organelles in cultured cells. Ddx4 1-236YFP (residues 1-236 conjugated to YFP) does not form organelles in cultured cells. However, when Ddx4 synth1_YFP and Ddx4 1-236CFP are expressed together, the truncated protein (Ddx4 1-236CFP) is recruited into the membraneless organelle.

FIG. 15|The solid, glassy state of the proteinaceous phase is an amorphous solid, produced by controlled evaporation of solvent (e.g. H₂O). Once formed, the glassy state of the proteinaceous phase can be physically picked up, moved, and deposited in a new location. The protein in the amorphous solid, glassy state can subsequently be re-dissolved through addition of solvent (e.g. H₂O). The process can be repeated multiple times.

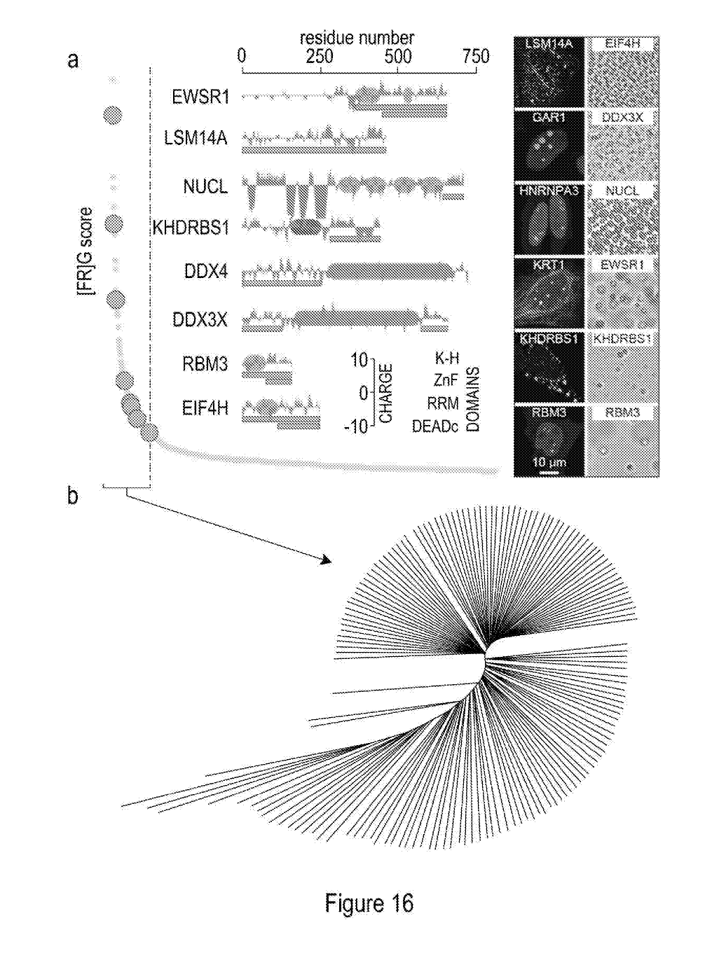

FIG. 16|Predicted and verified proteinaceous phase-forming proteins. a, Using a bioinformatics algorithm, potential proteinaceous phase-forming proteins were identified and ranked according to the predicted probability of undergoing phase separation akin to the prototypical Ddx4 protein. In total 1556 proteins were identified. The dashed line in panel ‘a’ indicates the top 10% of predicted proteins (156 proteins). Larger filled circles indicate the position on the ranked list of proteins verified to undergo phase separation to a liquid-like proteinaceous phase in vitro and in cells. Microscopy images show the manifestation of proteinaceous globules formed by the predicted proteins in cells and in vitro. b, Dendrogram produced by pairwise sorting (based on sequence identity) of the top 10% of bioinformatics hits.

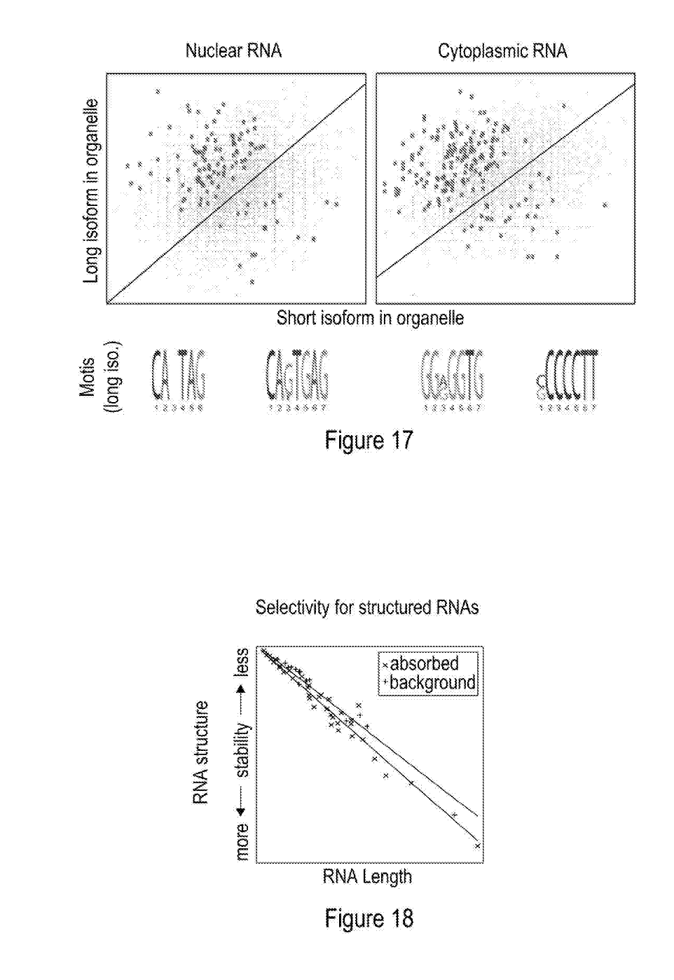

FIG. 17|For RNAs with alternative 5′UTRs that were preferentially absorbed by the organelle (large dots), longer isoforms were more frequently absorbed than the shorter isoforms. RNAs not significantly absorbed by the organelle phase are shown as small dots. Motif analysis showing an over-representation of G and C-rich motifs in preferentially absorbed long isoforms in cytoplasmic RNAs and CAxT motifs from nuclear RNAs.

FIG. 18|Longer and more highly structured RNAs were absorbed more strongly into the organelle phase.

### EXAMPLE: NATURALLY OCCURRING ORGANIC SOLVENTS INSIDE CELLS: MEMBRANELESS ORGANELLES MELT NUCLEIC ACID DUPLEXES AND ACT AS BIOMOLECULAR FILTERS

### Summary

Biochemical reactions inside cells are generally considered to occur in water. Cellular compartments termed ‘membraneless organelles’ challenge this view. These bodies are readily observable in the light microscope, for example nucleoli, Cajal bodies, P-bodies and nuage^{1,2} (FIG. 1). All can rapidly assemble and dissolve following changes in the cellular environment and cell cycle, and are predominantly associated with nucleic acid biochemistry. Membraneless organelles are liquid droplets³⁻⁵ formed by a phase separation of disordered proteins⁶⁻¹³, and offer a solvent environment distinct from the bulk aqueous phase that makes up the majority of the cellular interior. Organelles show similarity to solvents such as DMSO and acetonitrile in terms of their dielectric⁶. Here, we demonstrate that membraneless compartments can destabilise one of the most stable biological structures known, the DNA double helix, by up to 6 orders of magnitude while simultaneously stabilising the structures formed by single stranded nucleic acids such as regulatory RNAs. It is a common strategy in organic chemistry to utilise different solvents to influence the behaviour of molecules and reactions. These results reveal that biological organisms have also evolved this capability.

### Introduction

It is not obvious why forming droplets of proteinaceous solvent would be beneficial to an organism. Membraneless organelles can act as filters: nucleoli exclude bulk chromatin in the nucleus¹⁴ (FIGS. 1a and 5a) and stress granules, p-bodies and nuage concentrate RNAs in the cytoplasm in order to regulate their interactions and activities¹⁵. Model nuage organelles made of the disordered N-terminus of Ddx4 protein (Ddx4^{N1}) form bodies similar in appearance and characteristics to the nucleolus of cultured cells (FIGS. 1b and 5b). These bodies exclude chromatin, yet absorb single stranded DNA molecules⁶. Previously, we devised an algorithm to predict disordered protein sequences that should also form organelles with similar interiors⁶. Here, we produced the protein for three of these sequences, and all formed liquid droplets that resembled membraneless organelles (FIG. 5c). Moreover, all were observed to concentrate RNA (FIG. 5c).

We employed a recently developed confocal microscopy method to ascertain whether fluorescently labelled DNA or RNA oligonucleotides were absorbed or excluded from organelles in vitro⁶. We began by investigating how nucleic acid length affected partitioning within Ddx4^{N1} organelles using a series of 5′-Cy5 labelled DNA and RNA oligonucleotides derived from concatenated ACTG (DNA) or ACUG (RNA) repeats. Annealing the labelled strand with an equal amount of an unlabelled sense or antisense strand of the same length, produced either single(ss)- or double(ds)-stranded nucleic acids ranging in length from a 12mer to a 40mer. The nucleic acids were mixed with Ddx4^{N1} organelles and their relative partitioning, or solubility, was monitored using confocal fluorescence microscopy, and quantified as a partition free energy (ΔGₚₐᵣₜ, FIG. 1ci, section S.9). Unstructured ssRNA was partitioned into the organelle most strongly, followed by ssDNA in a manner that was largely independent of oligonucleotide length.

We next sought to investigate the partitioning of structured nucleic acid hairpins, containing a mixture of double-stranded (stem) and single-stranded (loop) regions. A series of 5′-Cy5 labelled DNAs and RNAs comprising 6 to 10 base pair (bp) GC stems and 20 base polyT/U loops displayed increased partitioning over the unstructured single stranded ACTG or ACUG sequences (FIG. 1 ciii). Short, regulatory RNAs such as miRNA, siRNA and piRNA are predicted to adopt hairpin conformations in solution, and are concentrated inside membraneless organelles such as nuage¹⁶ and RNA granules^{17,18}. We tested the partitioning of a mature miRNA (Let-7a)^{19,20,} and three nuage-associated and transposon-derived piRNAs^{21,22} (FIG. 1civ). These physiological RNA hairpins partitioned inside organelles more than the unstructured sequences of similar length.

By contrast to the case of single-stranded oligonucleotides, highly rigid double-stranded DNA, RNA, and hybrid RNA/DNA duplexes of 20 base pairs or longer were predominantly excluded from Ddx4 organelles (FIG. 1c, table 3). Intriguingly, the fluorescence signal from the 12mer and 16mer duplexes was enriched inside the organelle droplets, though not at the same level as their purely single-stranded equivalents. To understand this result in more detail, we used a Forster resonance energy transfer (FRET) assay to monitor the stability of the 12mer and 24mer DNA duplexes in the presence of the organelles. We added equimolar amounts of either a sense or antisense strand labelled with Cy3 to our original Cy5 ACTG oligonucleotides, to produce FRET-pair labelled ssDNA or dsDNA (FIG. 2ai). Both the partitioning (FIG. 2aii), and FRET (FIG. 2 aiii, section S.12), were measured inside and outside of the Ddx4^{N1} organelles. The single stranded 12mer exhibited low FRET both inside and outside organelles, indicating no association in either phase. Conversely, although the partitioning tendencies are opposed, both the 12mer and 24mer dsDNAs showed high FRET outside organelles, but markedly diminished FRET inside organelles (FIG. 2aiii, b). To put the observed reduction in FRET into context, we performed a denaturing titration of the 12mer dsDNA with guanidinium chloride (GdmHCl). The loss of FRET signal inside the organelles was consistent with that observed at highly denaturing conditions (4M GdmHCl, FIG. 2c) amounting to a destabilisation in the KD by 6 orders of magnitude. From these data, we conclude that the interior of Ddx4^{N1} organelles excludes nucleic acid duplexes, and solubilises single strands (FIG. 2d).

To move towards a mechanistic understanding of the partitioning, the partition free energy was found to vary strikingly as a function of the predicted stability (ΔG_{stab}) of the nucleic acid structures studied (FIG. 3a, table 3). The stabilisation is due to folding in the case of the hairpin structures (FIG. 3bi), and association in the case of double stranded oligonucleotides (FIG. 3bii). Nucleic acids with ΔGstab less than −125 kJ mol⁻¹ were partitioned inside, whereas those with greater ΔGstab were excluded. The data were analysed in terms of a thermodynamic model, which links the stability of the oligonucleotide structures inside and outside of the organelles to the observed portioning behaviour (FIG. 3bi/ii, section S.10). From this analysis, the ratio of double to single stranded oligonucleotides was lower inside the organelles than outside, supporting the results from the FRET analysis (FIG. 2). Similarly, as the partition free energy of single stranded RNA was less than that observed for structured, physiologically relevant RNAs, the secondary structure is effectively stabilised inside the organelles (FIG. 3c). Moreover, a quantitative analysis of how the thermodynamic parameters vary with overall number of base pairs revealed that the contribution to the partition free energies by individual base pairs within an oligonucleotide decrease as the sequence is lengthened (FIG. 3c (solid lines), FIG. 7c). This scaling is quantitatively consistent with ‘topological frustration’ inside the organelles, observed previously in amyloid fibrils²³ (section S.11). Taken together, the crowded interior of the organelle tends to favour compact, oligonucleotide structures over extended or rigid conformations (FIG. 3c, d), which can be explained in terms of the ease at which structures can fit into the mesh-like weave of Ddx4^{N1} proteins that form the interior of the organelle, without distorting any underlying structure.

To complement the characterisation of organelle partitioning of oligonucleotides, we mixed a range of fluorescently tagged proteins with organelles and measured their partition free energies (table 4). Fluorescence from isolated GFP and YFP was observed in approximately equal quantities both inside and outside of the organelle droplets indicating that they are folded in the organelle interior²⁴. Proteins conjugated to GFP, CFP or YFP showed a greater range of partitioning than we observed in the nucleotides (FIG. 4a, 8ai). A range of tagged variants of Ddx4 and PiwiL1, a nuage protein expressed during the latter stages of spermiogenesis, were all absorbed. By contrast, two fluorescently tagged small heat shock protein (HSP) oligomers of Hsp16.5 and αB-crystallin (average oligomer weights 1.1 and 1.4 MDa, respectively) were both strongly excluded from the organelles. The extent to which a protein is absorbed can be quantitatively described by knowledge of the proline, arginine and tryptophan content (FIG. 8bi, ii) suggesting the organelles naturally have a complex recognition scheme for protein entry and trafficking.

A series of GFP proteins were introduced that varied in surface charge²⁵. Both an acidic GFP (−30 surface charge, GFP⁻³⁰) and a basic GFP (+15 surface charge, GFP⁺¹⁵) were absorbed inside the organelles more than wild-type GFP (−7 surface charge, GFP^{WT}, FIG. 4a). Such supercharged GFP proteins have been used as delivery vehicles for various nucleic acids into live cells²⁶. We therefore tested whether GFP⁺¹⁵ could chaperone an otherwise excluded nucleic acid into Ddx4 organelles. For this we pre-incubated GFP^{WT} and GFP⁺¹⁵ with 40mer or 24mer dsDNA (FIG. 1c), and introduced the mixtures to Ddx4^{N1} organelles. Partitioning of GFP^{WT} and the dsDNAs were largely unchanged when premixed together (FIGS. 4bi, and 8aiii, iv). However, in the presence of GFP⁺¹⁵, the dsDNA partitioning was reversed, with DNA now being concentrated inside the organelles (FIG. 4bi, 8aiii, iv). FRET between the strands of the imported duplex was diminished inside the organelles, indicating that once chaperoned inside, the double stranded DNA was melted (FIG. 4bii, iii, 8c).

In further experiments, complex mixtures of RNA (with likely more than 10,000 unique RNA molecules ranging in length of 100-100,000 nucleotides long, and potentially hundreds of copies of each) were fractionated into populations that were either absorbed or excluded by phase-separated droplets of Ddx4 protein (organelle phase). The experiment was performed by forming organelles in the presence of complex RNA mixtures (by rapidly reducing the ionic strength from 300 to 100 mM NaCl), incubating the samples at 4° C. for 1 hour, collecting the organelle phase (˜0.5% total sample volume) at the bottom of an eppendorf tube by bench-top centrifugation (<1 min), aspirating the supernatant phase (˜99.5% total sample volume) and resuspending the organelle phase in a high ionic strength buffer (300 mM NaCl). This yielded two fractions per experiment containing RNAs that were either absorbed of excluded from the organelle phase. RNA in each fraction (absorbed or excluded) were subsequently subjected to deep sequencing in order to identify their sequences and quantitate their relative absorption/exclusion. The experiment was repeated with either nuclear RNA or cytoplasmic RNA as the starting material (constituting the complex mixtures of RNAs).

An analysis was performed in which the absorption or exclusion of RNAs with alternative 5′ untranslated regions (5′UTRs), differing in length by 100-10,000 nucleotides, was compared. This analysis revealed, for a given RNA with alternative 5′UTRs, whether the long isoform or the short isoform was preferentially absorbed into the organelle phase. In the majority of instances, when there was significant absorption into the organelle and for both nuclear and cytoplasmic RNA, the RNA molecule containing the longer 5′UTR was absorbed more strongly then the equivalent RNA bearing the short isoform (FIG. 17).

An analysis was then performed to identify short sequence motifs (7 nucleotides in length) contained in long 5′UTR isoforms and absent from the short 5′UTR isoform equivalent. Overall, for cytoplasmic RNAs, the preferentially absorbed 5′UTRs contained a high G and C content. Strongly absorbed nuclear RNAs contained an overrepresentation of CAxT motifs (where x is any of A, T, C or G nucleotides).

Further analysis of the alternative 5′UTR sequences that conferred preferential absorption to the organelle phase (sequences unique to the long isoforms) revealed that both length and predicted structure in the RNA was correlated with absorption. i.e. longer and more highly structured RNAs were more highly absorbed. FIG. 18.

### CONCLUSION

Overall, we have demonstrated that the interior of the organelle has very different properties to the bulk aqueous phase of the cell. Individual proteins and oligonucleotides exhibit a complex but predictable set of tendencies to enter the organelles. Moreover, double stranded DNA can be destabilised by up to 6 orders of magnitude without the need for ATP while single stranded structures can be simultaneously stabilised in a manner that suggests the interior of the organelle favours compact conformations.

### REFERENCES

1. Misteli, T. & Spector, D. L. The Nucleus: A Subject Collection from Cold Spring Harbor Perspectives in Biology. (Cold Spring Harbor Laboratory Press, 2011).
2. Voronina, E., Seydoux, G., Sassone-Corsi, P. & Nagamori, I. RNA Granules in Germ Cells. Csh Perspect Biol 3, (2011).
3. Brangwynne, C. P. et al. Germline P Granules Are Liquid Droplets That Localize by Controlled Dissolution/Condensation. Science 324, 1729-1732, (2009).
4. Brangwynne, C. P., Mitchison, T. J. & Hyman, A. A. Active liquid-like behavior of nucleoli determines their size and shape in Xenopus laevis oocytes. P Natl Acad Sci USA 108, 43344339, (2011).
5. Berry, J., Weber, S. C., Vaidya, N., Haataja, M. & Brangwynne, C. P. RNA transcription modulates phase transition-driven nuclear body assembly. P Natl Acad Sci USA, (2015).
6. Nott, T. J. et al. Phase Transition of a Disordered Nuage Protein Generates Environmentally Responsive Membraneless Organelles. Mol Cell 57, 936-947, (2015).
7. Li, P. L. et al. Phase transitions in the assembly of multivalent signalling proteins. Nature 483, 336-U129, (2012).
8. Elbaum-Garfinkle, S. et al. The disordered P granule protein LAF-1 drives phase separation into droplets with tunable viscosity and dynamics. P Natl Acad Sci USA 112, 7189-7194, (2015).
9. Weber, S. C. & Brangwynne, C. P. Inverse Size Scaling of the Nucleolus by a Concentration-Dependent Phase Transition. Curr Biol 25, 641-646, (2015).
10. Patel, A. et al. A Liquid-to-Solid Phase Transition of the ALS Protein FUS Accelerated by Disease Mutation. Cell 162, 1066-1077, (2015).
11. Lin, Y., Protter, D. S., Rosen, M. K. & Parker, R. Formation and Maturation of Phase-Separated Liquid Droplets by RNA-Binding Proteins. Mol Cell, (2015).
12. Molliex, A. et al. Phase Separation by Low Complexity Domains Promotes Stress Granule Assembly and Drives Pathological Fibrillization. Cell 163, 123-133, (2015).
13. Jiang, H. et al. Phase Transition of Spindle-Associated Protein Regulate Spindle Apparatus Assembly. Cell, (2015).
14. Swedlow, J. R. & Lamond, A. I. Nuclear dynamics: where genes are and how they got there. Genome Biol 2, (2001).
15. Anderson, P. & Kedersha, N. RNA granules: post-transcriptional and epigenetic modulators of gene expression. Nat Rev Mol Cell Bio 10, 430-436, (2009).
16. Meikar, O., Da Ros, M., Liljenback, H., Toppari, J. & Kotaja, N. Accumulation of piRNAs in the chromatoid bodies purified by a novel isolation protocol. Exp Cell Res 316, 1567-1575, (2010).
17. Han, T. N. W. et al. Cell-free Formation of RNA Granules: Bound RNAs Identify Features and Components of Cellular Assemblies. Cell 149, 768-779, (2012).
18. Kato, M. et al. Cell-free Formation of RNA Granules: Low Complexity Sequence Domains Form Dynamic Fibers within Hydrogels. Cell 149, 753-767, (2012).
19. Pasquinelli, A. E. et al. Conservation of the sequence and temporal expression of let-7 heterochronic regulatory RNA. Nature 408, 86-89, (2000).
20. Reinhart, B. J. et al. The 21-nucleotide let-7 RNA regulates developmental timing in Caenorhabditis elegans. Nature 403, 901-906, (2000).
21. Aravin, A. A. et al. A piRNA pathway primed by individual transposons is linked to de novo DNA methylation in mice. Mol Cell 31, 785-799, (2008).
22. Watanabe, T. et al. Identification and characterization of two novel classes of small RNAs in the mouse germline: retrotransposon-derived siRNAs in oocytes and germline small RNAs in testes. Gene Dev 20, 1732-1743, (2006).
23. Baldwin, A. J. et al. Metastability of Native Proteins and the Phenomenon of Amyloid Formation. J Am Chem Soc 133, 14160-14163, (2011).
24. Craggs, T. D. Green fluorescent protein: structure, folding and chromophore maturation. Chem Soc Rev 38, 2865-2875, (2009).
25. Lawrence, M. S., Phillips, K. J. & Liu, D. R. Supercharging proteins can impart unusual resilience. J Am Chem Soc 129, 10110-10112, (2007).
26. McNaughton, B. R., Cronican, J. J., Thompson, D. B. & Liu, D. R. Mammalian cell penetration, siRNA transfection, and DNA transfection by supercharged proteins. P Natl Acad Sci USA 106, 6111-6116, (2009).

### Supplementary Information

### Materials and Methods.

### S.1 HeLa Cell Culture¹

HeLa cells were cultured as previously described¹. Briefly, cells were grown on 25 mm glass coverslips in growth media (high glucose DMEM containing 20 mM HEPES pH 7.4, 10% FBS and antibiotics at 37° C. and 5% CO2). Ddx4 constructs (Ddx4^{YFP} and Ddx4^{YFP-FtoA}) were expressed in HeLa cells from pcDNA 3.1+(Invitrogen) plasmids by transient transfection utilizing the Effectene (Qiagen) or polyethylenimine (PEI) methods. Transfections were carried out according to the manufacturer's instructions and used 0.5-1 μg plasmid DNA per coverslip.

### S.2 Imaging Fixed HeLa Cells¹

HeLa cells expressing Ddx4^{YFP} and Ddx4^{YFP-FtoA} were grown on 25 mm glass coverslips, and fixed with 4% paraformaldehyde (PFA) in phosphate buffered saline (PBS), for 5 minutes at 37° C. Cells were then washed three times with PBS to remove excess PFA. Next, cells were permeabilised with 0.5% TritonX-100 (in PBS) for 10 minutes, and again washed three times with PBS. Nuclei were visualized with Hoechst or DAPI stain. Cells were washed a further two times with PBS to remove excess Hoechst/DAPI stain and imaged using an Olympus IX81 inverted microscope with a 60× (NA 1.3) silicon immersion objective. Hoechst/DAPI dye was excited with a 405 nm laser and YFP was excited with a 515 nm laser. Hoechst/DAPI and YFP fluorescence were detected at 461 and 527 nm respectively. Differential interference contrast (DIC) images were collected using illumination from the 405 nm laser.

### S.3 Immunofluorescence¹

For immunofluorescence experiments, HeLa cells expressing Ddx4^{YFP} were grown on 25 mm diameter #1.5 glass coverslips (Warner Instruments). The fixation and permeabilisation of samples was performed as above. Cells were then blocked with goat serum (5% in PBS) for one hour at room temperature before antibody staining. Primary antibodies were diluted to between 1:10 and 1:100 (in PBS containing 5% goat serum) before use. Nucleoli were labelled using mouse B23 (Santa Cruz sc-56622) antibodies. Following incubation at 4° C. overnight, excess primary antibodies were removed by washing the cells three times with PBS (five minutes per wash).

Cells were then incubated with Cy3 goat anti-mouse secondary antibodies (diluted 1:400) for 1 hour at room temperature. Excess secondary antibodies were removed by washing with PBS, as above. Nuclei were visualized with DAPI stain. Coverslips were then mounted on microscope slides using glycerol/n-propyl gallate mounting medium, sealed with nail varnish, and imaged using an Olympus IX81 inverted microscope equipped with a 60×(NA 1.3) silicon immersion objective. DAPI, YFP and Cy3 and dyes were excited with 405 nm, 515 nm and 559 nm lasers, respectively. 26 Z-slices (0.4 μm spacing, 12.5 μs pixel⁻¹ scanning speed, 12 bit depth) were captured for each channel.

### S.4 Protein Expression and Purification

Recombinant proteins for both organelle formation and partitioning were expressed from IPTG-inducible plasmids in E. coli cells overnight at 20° C. Cell pellets were suspended in buffer (50 mM Tris pH 8.0, 500 mM NaCl, 5 mM DTT) and lysed by homogenisation. Proteins were first purified by affinity chromatography (GST-4b beads, GE Healthcare Life Sciences or Ni-NTA, Invitrogen). The GST tag was removed with TEV-protease, and the target protein further purified and buffer exchanged by size exclusion chromatography into storage buffer (20 mM Tris pH 8.0, 300 mM NaCl, 5 mM TCEP). Purified proteins were centrifugally concentrated and flash frozen in liquid nitrogen and stored at −80° C. The sequences of all proteins used in this study are summarised in table 2.

TABLE 2
Summary of protein sequences used in this study.

| | |
|---|---|
| Ddx4_N1_1 | GAMGSMGDEDWEAEINPHMSSYVPIFEKDRYSGENGDNFNRTP |
| | ASSSEMDDGPSRRDHFMKSGFASGRNFGNRDAGECNKRDNTST |
| | MGGFGVGKSFGNRGFSNSRFEDGDSSGFWRESSNDCEDNPTRN |
| | RGFSKRGGYRDGNNSEASGPYRRGGRGSFRGCRGGFGLGSPNN |
| | DLDPDECMQRTGGLFGSRRPVLSGTGNGDTSQSRSGSGSERGG |
| | YKGLNEEVITGSGKNSWKSEAEGGESSDTQGPKVT |
| | (SEQ ID NO: 1) |
| GFP^{WT} | MHHHHHHMSKGEELFTGVVPILVELDGDVNGHKFSVRGEGEG |
| | DATNGKLTLKFICTTGKLPVPWPTLVTTLGYGVQCFSRYPDHM |
| | KRHDFFKSAMPEGYVQERTISFKDDGTYKTRAEVKFEGDTLVN |
| | RIELKGIDFKEDGNILGHKLEYNFNSHNVYITADKQKNGIKANF |
| | KIRHNVEDGSVQLADHYQQNTPIGDGPVLLPDNHYLSTQSVLS |
| | KDPNEKRDHMV LLEFVTAAGITHG |
| | (SEQ ID NO: 2) |
| GFP⁺¹⁵ | MHHHHHHMSKGERLFTGVVPILVELDGDVNGHKFSVRGEGEG |
| | DATRGKLTLKFICTTGKLPVPWPTLVTTLTYGVQCFSRYPKHM |
| | KRHDFFKSAMPEGYVQERTISFKKDGTYKTRAEVKFEGRTLVN |
| | RIELKGRDFKEKGNILGHKLEYNFNSHNVYITADKRKNGIKANF |
| | KIRHNVKDGSVQLADHYQQNTPIGRGPVLLPRNHYLSTRSALS |
| | KDPKEKRDHMV LLEFVTAAGITHGMDELYK |
| | (SEQ ID NO: 3) |
| GFP⁻³⁰ | MHHHHHHMSKGEELFDGVVPILVELDGDVNGHEFSVRGEGEG |
| | DATEGELTLKFICTTGELPVPWPTLVTTLTYGVQCFSDYPDHMD |
| | QHDFFKSAMPEGYVQERTISFKDDGTYKTRAEVKFEGDTLVNR |
| | IELKGIDFKEDGNILGHKLEYNFNSHDVYITADKQENGIKAEFEI |
| | RHNVEDGSVQLADHYQQNTPIGDGPVLLPDDHYLSTESALSKD |
| | PNEDRDHMV LLEFVTAAGIDHGMDELYK |
| | (SEQ ID NO: 4) |
| hDdx4_1- | GAMGSMGDEDWEAEINPHMSSYVPIFEKDRYSGENGDNFNRTP |
| 236_YFP | ASSSEMDDGPSRRDHFMKSGFASGRNFGNRDAGECNKRDNTST |
| | MGGFGVGKSFGNRGFSNSRFEDGDSSGFWRESSNDCEDNPTRN |
| | RGFSKRGGYRDGNNSEASGPYRRGGRGSFRGCRGGFGLGSPNN |
| | DLDPDECMQRTGGLFGSRRPVLSGTGNGDTSQSRSGSGSERGG |
| | YKGLNEEVITGSGKNSWKSEAEGGESVDMVSKGEELFTGVVPI |
| | LVELDGDVNGHKFSVSGEGEGDATYGKLTLKFICTTGKLPVPW |
| | PTLVTTFGYGLMCFARYPDHMKQHDFFKSAMPEGYVQERTIFF |
| | KDDGNYKTRAEVKFEGDTLVNRIELKGIDFKEDGNILGHKLEY |
| | NYNSHNVYIMADKQKNGIKVNFKIRHNIEDGSVQLADHYQQNT |
| | PIGDGPVLLPDNHYLSYQSKLS |
| | KDPNEKRDHMVLLEFVTAAGIT |
| | (SEQ ID NO: 5) |
| hDdx4_synth_1_Y | GAMGSNMGDEDWEAEINPHMSSYVPIFEKDRYSGENGDNFNR |
| FP_in_pETM30_2 | TPASSSEMDDGPSRRDHFMKSGFASGRNFGNRDAGECNKRDNT |
| HTb | STMGGFGVGKSFGNRGFSNSRFEDGDSSGFWRESSNDCEDNPT |
| | RNRGFSKRGGYRDGNNSEASGPYRRGGRGSFRGCRGGFGLGSP |
| | NNDLDPDECMQRTGGLFGSRRPVLSGTGNGDTSQSRSGSGSER |
| | GGYKGLNEEVITGSGKNSWKSEAEGGESSDTQGPKVTLQMVSK |
| | GEELFTGVVPILVELDGDVNGHKFSVSGEGEGDATYGKLTLKFI |
| | CTTGKLPVPWPTLVTTFGYGLMCFARYPDHMKQHDFFKSAMP |
| | EGYVQERTIFFKDDGNYKTRAEVKFEGDTLVNRIELKGIDFKED |
| | GNILGHKLEYNYNSHNVYIMADKQKNGIKVNFKIRHNIEDGSV |
| | QLADHYQQNTPIGDGPVLLPDNHYLSYQSKLSKDPNEKRDHMV |
| | LLEFVTAAGITLEFSTYIPGFSGSTRGNVFASVDTRKGKSTLNTA |
| | GFSSSQAPNPVDDESWD |
| | (SEQ ID NO: 6) |
| hDdx4_synth_4_Y | GAMGSNMGDEDWEAEINPHMSSYVPIFEKDRYSGENGDNFNR |
| FP_in_pETM30_2 | TPASSSEMDDGPSRRDHFMKSGAASGRNAGNRDAGECNKRDN |
| HTb | TSTMGGAGVGKSAGNRGASNSRFEDGDSSGFWRESSNDCEDNP |
| | TRNRGASKRGGYRDGNNSEASGPYRRGGRGSARGCRGGAGLG |
| | SPNNDLDPDECMQRTGGLAGSRRPVLSGTGNGDTSQSRSGSGS |
| | ERGGYKGLNEEVITGSGKNSWKSEAEGGESSDTQGPKVTLQMV |
| | SKGEELFTGVVPILVELDGDVNGHKFSVSGEGEGDATYGKLTL |
| | KFICTTGKLPVPWPTLVTTFGYGLMCFARYPDHMKQHDFFKSA |
| | MPEGYVQERTIFFKDDGNYKTRAEVKFEGDTLVNRIELKGIDFK |
| | EDGNILGHKLEYNYNSHNVYIMADKQKNGIKVNFKIRHNIEDG |
| | SVQLADHYQQNTPIGDGPVLLPDNHYLSYQSKLSKDPNEKRDH |
| | MVLLEFVTAAGITLEASTYIPGASGSTRGNVFASVDTRKGKSTL |
| | NTAGFSSSQAPNPVDDESWD |
| | (SEQ ID NO: 7) |
| YFP | GAMGSMVSKGEELFTGVVPILVELDGDVNGHKFSVSGEGEGDA |
| | TYGKLTLKFICTTGKLPVPWPTLVTTFGYGLMCFARYPDHMKQ |
| | HDFFKSAMPEGYVQERTIFFKDDGNYKTRAEVKFEGDTLVNRI |
| | ELKGIDFKEDGNILGHKLEYNYNSHNVYIMADKQKNGIKVNFK |
| | IRHNIEDGSVQLADHYQQNTPIGDGPVLLPDNHYLSYQSKLSKD |
| | PNEKRDHMVL |
| | LEFVTAAGITLERPLSNLEPAVSRHAVPSFKLSNN |
| | (SEQ ID NO: 8) |
| sHSP16.5_GFP | MFGRDPFDSLFERMFKEFFATPMTGTTMIQSSTGIQISGKGFMPI |
| | SIIEGDQHIKVIAWLPGVNKEDIILNAVGDTLEIRAKRSPLMITES |
| | ERIIYSEIPEEEEIYRTIKLPATVKEENASAKFENGVLSVILPKAES |
| | SIKKGINIEASGENLYFQSLSKGEELFTGVVPILVELDGDVNGHK |
| | FSVRGEGEGDATNGKLTLKFICTTGKLPVPWPTLVTTLTYGVQC |
| | FSRYPDHMKQHDFFKSAMPEGYVQERTISFKDDGTYKTRAEVK |
| | FEGDTLVNRIELKGIDFKEDGNILGHKLEYNFNSHNVYITADKQ |
| | KNGIKANFKIRHNVEDGSVQLADHYQQNTPIGDGPVLLPDNHY |
| | LSTQSVLSKDPNEKRDHMVLLEFVTAAGITHGMDELYKKGLEH |
| | HHHHH |
| | (SEQ ID NO: 9) |
| aB_GFP | MDIAIHHPWIRRPFFPFHSPSRLFDQFFGEHLLESDLFPTSTSLSP |
| | FYLRPPSFLRAPSWFDTGLSEMRLEKDRFSVNLDVKHFSPEELK |
| | VKVLGDVIEVHGKHEERQDEHGFISREFHRKYRIPADVDPLTITS |
| | SLSSDGVLTVNGPRKQVSGPERTIPITREEKPAVTAAPKKASGE |
| | NLYFQSLSKGEELFTGVVPILVELDGDVNGHKFSVRGEGEGDA |
| | TNGKLTLKFICTTGKLPVPWPTLVTTLTYGVQCFSRYPDHMKQ |
| | HDFFKSAMPEGYVQERTISFKDDGTYKTRAEVKFEGDTLVNRIE |
| | LKGIDFKEDGNILGHKLEYNFNSHNVYITADKQKNGIKANFKIR |
| | HNVEDGSVQLADHYQQNTPIGDGPVLLPDNHYLSTQSVLSKDP |
| | NEKRDHMVLLEFVTAAGITHGMDELYKKGLEHHHHHH |
| | (SEQ ID NO: 10) |
| hPiwiL1_1-109 | CFPFVTAAGITGAMGSMTGRARARARGRARGQETAQLVGSTAS |
| | QQPGYIQPRPQPPPAEGELFGRGRQRGTAGGTAKSQGLQISAGF |
| | QELSLAERGGRRRDFHDLGVNTRQNLDHVKESKTGSSGVDMV |
| | SKGEELFTGVVPILVELDGDVNGHKFSVSGEGEGDATYGKLTL |
| | KFICTTGKLPVPWPTLVTTLTWGVQCFARYPDHMKQHDFFKSA |
| | MPEGYVQERTIFFKDDGNYKTRAEVKFEGDTLVNRIELKGIDFK |
| | EDGNILGHKLEYNAISDNVYITADKQKNGIKANFKIRHNIEDGS |
| | VQLADHYQQNTPIGDGPVLLPDNHYLSTQSKLSKDPNEKRDHM |
| | VLLE |
| | (SEQ ID NO: 11) |

### S.5 Oligonucleotide Sample Preparation

DNA and RNA oligonucleotides, including those combined with the fluorescent Cy3 and Cy5 dyes, were purchased from SIGMA and delivered as lyophilised samples. Stocks at 100 μM were made by resuspending the oligonucleotides in TE buffer (10 mM Tris pH 8.0 at RT, 1 mM EDTA) and stored at −20° C. Lower concentration working stocks were made by further dilution with TE buffer.

Prior to partitioning experiments, oligonucleotides mixtures (sense+sense or sense+antisense) were heated to >95° C. for 2 minutes, and allowed to cool to room temperature over the course of 90-120 minutes. Hairpin oligonucleotides were heated to >95° C. for 2 minutes and snap-cooled on ice before equilibrating at room temperature for 5 minutes.

TABLE 3
Summary of sequence identity, predicted stabilities and experimentally
determined partition free energies of the oligonucleotides studied
here (FIG. 1, 3). The uncertainties in the experimentally derived
partition free energies ΔGpart, err, were obtained by taking the
standard deviation of the partitioning of 60 organelles (5 per field
of view (FOV), 3 FOVs per sample, 2 samples) per oligonucleotide, as
described below.

| | | ΔGstab, | ΔGstab, | | ΔGpart, | sequence | sequence |
|---|---|---|---|---|---|---|---|
| | Length | ds_ | ss_ | ΔGpart | err | (sense | (additional |
| Label | (bp) | (kJmol⁻¹) | kJmol⁻¹) | (kJmol⁻¹) | kJmol⁻¹) | strand) | strand) |
| dsDNA_ACT | 12 | −57.8 | +15.4 | 2.67 | 0.57 | [Cy5]ACTGACTG | CAGTCAGTCAGT |
| G | | (−64.04 ± | | | | ACTG | (SEQ ID |
| | | 4.89)- | | | | (SEQ ID NO: 12) | NO: 13) |
| | | 81.6 | | | | | |
| dsDNA_ACT | 16 | (−75.43 ± | +15.1 | 3.93 | 0.65 | [Cy5]ACTGACTG | CAGTCAGTCAGT |
| G | | 7.82) | | | | ACTGACTG | CAGT |
| | | | | | | (SEQ ID NO: 14) | (SEQ ID |
| | | | | | | | NO: 15) |
| dsDNA_ACT | 20 | −105.5 | +15.1 | −0.95 | −0.28 | [Cy5]ACTGACTG | CAGTCAGTCAGT |
| G | | (−132.35 ± | | | | ACTGACTGACTG | CAGTCAGT |
| | | 20.63) | | | | (SEQ ID NO: 16) | (SEQ ID |
| | | | | | | | NO: 17) |
| dsDNA_ACT | 24 | −129.3 | +15.1 | −1.88 | −0.36 | [Cy5]ACTGACTG | CAGTCAGTCAGT |
| G | | | | | | ACTGACTGACTG | CAGTCAGTCAGT |
| | | | | | | (SEQ ID NO: 18) | (SEQ ID |
| | | | | | | | NO: 19) |
| dsDNA_ACT | 28 | −153.2 | +15.1 | −1.75 | −1.33 | ACTG[Cy5]ACTGAC | CAGTCAGTCAGT |
| G | | | | | | TGACTGACTGAC | CAGTCAGTCAGT |
| | | | | | | TGACTGACTG | CAGT |
| | | | | | | (SEQ ID NO: 20) | (SEQ ID |
| | | | | | | | NO: 21) |
| dsDNA_ACT | 32 | −177.0 | +15.1 | −2.55 | −0.49 | [Cy5]ACTGACTG | CAGTCAGTCAGT |
| G | | | | | | ACTGACTGACTG | CAGTCAGTCAGT |
| | | | | | | ACTGACTGACTG | CAGTCAGT |
| | | | | | | (SEQ ID NO: 22) | (SEQ ID |
| | | | | | | | NO: 23) |
| dsDNA_ACT | 36 | −200.9 | +15.1 | −2.42 | −0.68 | [Cy5]ACTGACTG | CAGTCAGTCAGT |
| G | | | | | | ACTGACTGACTG | CAGTCAGTCAGT |
| | | | | | | ACTGACTGACTG | CAGTCAGTCAGT |
| | | | | | | ACTG | (SEQ ID |
| | | | | | | (SEQ ID NO: 24) | NO: 25) |
| dsDNA_ACT | 40 | −224.7 | +15.1 | −2.99 | −0.39 | [Cy5]ACTGACTG | CAGTCAGTCAGT |
| G | | | | | | ACTGACTGACTG | CAGTCAGTCAGT |
| | | | | | | ACTGACTGACTG | CAGTCAGTCAGT |
| | | | | | | ACTGACTG | CAGT |
| | | | | | | (SEQ ID NO: 26) | (SEQ ID |
| | | | | | | | NO: 27) |
| ssDNA_ACT | 12 | +9.9 | +15.4 | 7.55 | 0.39 | [Cy5]ACTGACTG | ACTGACTGACTG |
| G | | | | | | ACTG | (SEQ ID |
| | | | | | | (SEQ ID NO: 28) | NO: 29) |
| ssDNA_ACT | 16 | +9.9 | +15.1 | 7.04 | 0.83 | [Cy5]ACTGACTG | ACTGACTGACTG |
| G | | | | | | ACTGACTG | ACTG |
| | | | | | | (SEQ ID NO: 30) | (SEQ ID |
| | | | | | | | NO: 31) |
| ssDNA_ACT | 20 | +9.9 | +15.1 | 7.27 | 0.33 | [Cy5]ACTGACTG | ACTGACTGACTG |
| G | | | | | | ACTGACTGACTG | ACTGACTG |
| | | | | | | (SEQ ID NO: 32) | (SEQ ID |
| | | | | | | | NO: 33) |
| ssDNA_ACT | 24 | +9.9 | +15.1 | 5.87 | 0.59 | [Cy5]ACTGACTG | ACTGACTGACTG |
| G | | | | | | ACTGACTGACTG | ACTGACTGACTG |
| | | | | | | ACTG | (SEQ ID |
| | | | | | | (SEQ ID NO: 34) | NO: 35) |
| ssDNA_ACT | 28 | +9.9 | +15.1 | 7.20 | 0.47 | [Cy5]ACTGACTG | ACTGACTGACTG |
| G | | | | | | ACTGACTGACTG | ACTGACTGACTG |
| | | | | | | ACTGACTG | ACTG |
| | | | | | | (SEQ ID NO: 36) | (SEQ ID |
| | | | | | | | NO: 37) |
| ssDNA_ACT | 32 | +9.9 | +15.1 | 6.71 | 0.31 | [Cy5]ACTGACTG | ACTGACTGACTG |
| G | | | | | | ACTGACTGACTG | ACTGACTGACTG |
| | | | | | | ACTGACTGACTG | ACTGACTG |
| | | | | | | (SEQ ID NO: 38) | (SEQ ID |
| | | | | | | | NO: 39) |
| ssDNA_ACT | 36 | +9.9 | +15.1 | 6.73 | 0.29 | [Cy5]ACTGACTG | ACTGACTGACTG |
| G | | | | | | ACTGACTGACTG | ACTGACTGACTG |
| | | | | | | ACTGACTGACTG | ACTGACTGACTG |
| | | | | | | ACTG | (SEQ ID |
| | | | | | | (SEQ ID NO: 40) | NO: 41) |
| ssDNA_ACT | 40 | +9.9 | +15.1 | 5.56 | 0.44 | [Cy5]ACTGACTG | ACTGACTGACTG |
| G | | | | | | ACTGACTGACTG | ACTGACTGACTG |
| | | | | | | ACTGACTGACTG | ACTGACTGACTG |
| | | | | | | ACTGACTG | ACTG |
| | | | | | | (SEQ ID NO: 42) | (SEQ ID |
| | | | | | | | NO: 43) |
| dsRNA_ACU | 12 | −87.6 | +7.6 | 2.41 | 0.14 | [Cy5]ACUGACUG | CAGUCAGUCAGU |
| G | | | | | | ACUG | (SEQ ID |
| | | | | | | (SEQ ID NO: 44) | NO: 45) |
| dsRNA_ACU | 24 | −203.2 | +7.6 | −2.13 | 0.14 | [Cy5]ACUGACUG | CAGUCAGUCAGU |
| G | | | | | | ACUGACUGACU | CAGUCAGUCA GU |
| | | | | | | GACUG | (SEQ ID |
| | | | | | | (SEQ ID NO: 46) | NO: 47) |
| dsRNA/DNA_ | 24 | −203.2 | +7.6 | −2.21 | 0.07 | [Cy5]ACUGACUG | CAGTCAGTCAGT |
| hybrid | | | | | | ACUGACUGACU | CAGTCAGTCAGT |
| | | | | | | GACUG | (SEQ ID |
| | | | | | | (SEQ ID NO: 48) | NO: 49) |
| ssRNA_ACU | 12 | +7.9 | +7.6 | 8.69 | 0.12 | [Cy5]ACUGACUG | ACUGACUGACUG |
| G | | | | | | ACUG | (SEQ ID |
| | | | | | | (SEQ ID NO: 50) | NO: 51) |
| ssRNA_ACU | 24 | +7.9 | +7.6 | 9.06 | 0.38 | [Cy5]ACUGACUG | ACUGACUGACUG |
| G | | | | | | ACUGACUGACU | ACUGACUGAC UG |
| | | | | | | GACUG | (SEQ ID |
| | | | | | | (SEQ ID NO: 52) | NO: 53) |
| 6bpDNA_ | 32 | −40.3 | −28.68 | 8.81 | 0.75 | [Cy3]GGCGGCTT | |
| hairpin | | | | | | TTTTTTTTTTTTT | |
| | | | | | | TTTTTGCCGCC | |
| | | | | | | [Cy5] | |
| | | | | | | (SEQ ID NO: 54) | |
| 6bpRNA_ | 32 | −56.5 | −50.28 | 9.79 | 1.72 | [Cy3]GGCGGCUU | |
| hairpin | | | | | | UUUUUUUUUUUU | |
| | | | | | | UUUUUUGCCGCC | |
| | | | | | | [Cy5] | |
| | | | | | | (SEQ ID NO: 55) | |
| 8bpDNA_ | 36 | −58.1 | −46.22 | 8.72 | 0.98 | [Cy3]GGCGGCGG | |
| hairpin | | | | | | TTTTTTTTTTTTT | |
| | | | | | | TTTTTTTCCGCC | |
| | | | | | | GCC[Cy5] | |
| | | | | | | (SEQ ID NO: 56) | |
| 10bpDNA_ | 40 | −95.0 | −65.52 | 7.44 | 0.70 | [Cy3]GGCGGCGG | |
| hairpin | | | | | | CGTTTTTTTTTTT | |
| | | | | | | TTTTTTTTTCGC | |
| | | | | | | CGCCGCC[Cy5] | |
| | | | | | | (SEQ ID NO: 57) | |
| Let- | 22 | −16.9 | −4.44 | 11.05 | 0.59 | [Cy5]UGAGGUAG | |
| 7a_RNA_ | | | | | | UAGGUUGUAU | |
| hairpin | | | | | | AGUU | |
| | | | | | | (SEQ ID NO: 58) | |
| IAP_ | 28 | −33.7 | −9.67 | 9.81 | 0.18 | [Cy5]UACCACUU | |
| 28_RNA_ | | | | | | AGAACACAGGAU | |
| hairpin | | | | | | GUCAGCGC | |
| | | | | | | (SEQ ID NO: 59) | |
| MT2_ | 30 | −76.0 | −23.19 | 9.94 | 0.21 | [Cy5]UGUGAAUG | |
| 30_RNA_ | | | | | | GAAGUCCAAGGA | |
| hairpin | | | | | | UCUAGCAGUU | |
| | | | | | | (SEQ ID NO: 60) | |
| LINE1_ | 32 | −57.2 | −28.89 | 7.51 | 0.15 | [Cy5]UGAAACUC | |
| 32_RNA_ | | | | | | CAAAGUUUCUCC | |
| hairpin | | | | | | AAGGCAAAAGAC | |
| | | | | | | (SEQ ID NO: 61) | |

### S.6 Theoretical Determination of Oligonucleotide Stability (ΔGstab)

Oligonucleotide stability (ΔGstab) was predicted using the calculator UNAFold² with 1 oligo (selecting DNA or RNA as appropriate), 150 mM NaCl at 25° C. (table 3). The stabilities of the 12mer, 16mer and 20mer ACTG duplexes were independently verified by experimental measurement in-house (section S.7).

### S.7 Experimental Determination of Oligonucleotide Stability (ΔGstab)

To compare with the theoretical estimates, the stability of the duplexes formed by the 12mer, 16mer and 20mer ACTG dsDNA samples were measured using a Chirascan circular dichroism spectrophotometer (Applied Photophysics), equipped with Series 800 Temperature Controller (AlphaOmega Instruments). Oligonucleotide samples were first heated to at least 85° C. for 5 minutes, followed by cooling at 1° C./min to 20° C. Absorbance at 278 nm was monitored (1 nm bandwidth, 1° C. step size, 0.5 sec per time point, 8 repeats) as the samples were cooled. The temperature at which the samples contained 50 duplex were derived from the decrease in hyperchromicity as the oligonucleotides annealed. Annealing profiles were measured at 4 concentrations per oligonucleotide sample, from which it was possible to derive ΔGstab.

The hyperchromicity was normalised to go from 1 at low temperatures (fully duplex) and 0 at high temperatures (no duplex), and taken to be mole fraction of duplex F=2[ds]/sstₒt where [ds] and sstot are the double stranded concentration and total concentration of all single chained nucleotides in the system. Noting that sstₒt=2[ds]+[ss], where [ss] is the total concentration of free single stranded DNA, the mole fraction F can be converted into a free energy:

$$Δ G = - RT ln \frac{[ ds ]}{{[ ss / 2 ]}^{2}} = - RT ln \frac{2 F}{{{ss}_{tot} ( 1 - F )}^{2}}$$

which varies with temperature as ΔG=ΔH−TΔS. Rearranging and solving for F gives its functional variance with temperature: F=1+e^{(ΔH/T-ΔS)/R}/ssₜₒₜ+{square root over ((1+e^{(ΔH/T-Δ5)/R}/ssₜₒₜ)²−1)} At the temperature midpoint, T_{M}, F=0.5 and so:

$$Δ G = Δ H - {T}_{m} Δ S = - {RT}_{m} ln \frac{4}{{ss}_{tot}}$$

A plot of 1/TM versus ln(4/sstₒt) enables determination of AH and AS. This method was applied to 12, 16 and 20mer duplex DNAs (FIG. 3a, x-error bars, FIG. 7a, b). The experimentally derived values were in excellent accord with those calculated from the sequence alone².

### S.8 Partitioning Experiments (Δ Gpart)

Ddx4 membraneless organelle samples were prepared as squashed drops as previously described¹. Briefly, 1.35 μL of Ddx4N1 (325 μM in GF buffer; 20 mM Tris pH 8.0 at RT, 300 mM NaCl, 5 mM TCEP) was mixed 1:1 with an oligo or fluorescent protein sample on a round 22 mm siliconised coverslip (Hampton Research) and equilibrated as a hanging drop over a well solution of 20 mM Tris pH 8 at RT, 150 mM NaCl for 15 minutes (30 minutes for ternary mixtures also containing GFP). The well was sealed with Vaseline. The coverslip was removed after the equilibration period, excess Vaseline removed with a 200 uL pipette tip, and the droplet dispersed onto a microscope slide (Fisherbrand Superfrost catalogue #12-550-123).

In all cases the final concentration of Cy5- and/or Cy3-labelled oligonucleotides in the hanging droplet was 1 μM (0.5 μM for ternary mixtures also containing GFPs (FIG. 4b, FIG. 8a)). For example, the dsDNA ACTG FRET sample contained the sense strand ([Cy5]ACTGACTGACTG—SEQ ID NO: 12) at 1 μM and the antisense strand (CAGTCAGTCAGT[Cy3]—SEQ ID NO: 13) at 1 μM. The protein portioning experiments were performed in an identical fashion. The final concentrations in the hanging drops are summarised in table 4 together with properties of the sequence.

TABLE 4
Summary of concentrations of proteins used in partitioning experiments and
sequence properties. The experimental free energies are shown together with a result
derived from the equation AG_{ca}l_{c} = a1Pro %] + 131Arg %] + z1Tyr %] where a = −0.524,
13 = 0.297 and z = 0.392 kJmol⁻¹ were optimised co-factors (FIG. 8a).

| | Conc | | | | | | | | | | | |
|---|---|---|---|---|---|---|---|---|---|---|---|---|
| | in | | | AGpart, | NaCl | | Monomer | Oligomer | | | | |
| | droplet | AGpart | AGpart, | calc | conc | length | MW | MW | | Pro | Arg | Tyr |
| | (μM) | (kJmol⁻1₎ | err kJmol⁻1₎ | (kJmol− | (mM) | (aa) | (kDa) | (kDa) | PI | (%) | (%) | (%) |
| Ddx4N1 | 162.5 | | | | | 250 | 26.7 | — | 5.43 | 4.0 | 9.6 | 2.0 |
| Ddx4N1YFP | 10 | 9.4 | 0.1 | 11.0 | 150 | 474 | 52.1 | — | 5.59 | 4.0 | 6.3 | 3.4 |
| Ddx4YFP | 10 | 9.1 | 0.1 | 7.6 | 150 | 535 | 58.6 | — | 5.54 | 4.3 | 6.0 | 3.2 |
| Ddx4YFP_FtoA | 10 | 5.5 | 0.1 | 7.6 | 150 | 535 | 57.8 | — | 5.54 | 4.3 | 6.0 | 3.2 |
| PiwiL1 CFP | 10 | 5.9 | 1.0 | 3.8 | 150 | 347 | 38.1 | — | 7.91 | 4.6 | 6.1 | 2.6 |
| YFP | 10 | 1.2 | 0.1 | −4.6 | 150 | 261 | 29.3 | — | 6.07 | 5.0 | 3.1 | 4.2 |
| GFP^{WT} | 5 | −2.9 | 0.1 | 1.4 | 150 | 239 | 26.9 | — | 6.33 | 4.2 | 3.3 | 3.3 |
| GFP⁻³⁰ | 5 | 1.6 | 0.2 | 0.7 | 150 | 245 | 27.7 | — | 4.64 | 4.1 | 2.4 | 3.7 |
| GFP⁺¹⁵ | 5 | 13.5 | 0.5 | 12.4 | 150 | 245 | 28.1 | — | 9.75 | 4.1 | 6.5 | 3.7 |
| aB-GFP | 11.8 | −7.8 | 0.7 | −7.6 | 175 | 433 | 49.2 | 1,378 | 6.27 | 6.2 | 4.8 | 2.8 |
| sHSP16.5- | 17.3 | −3.4 | 0.3 | −2.0 | 175 | 405 | 45.6 | 1,092 | 5.73 | 4.4 | 3.2 | 3.0 |

### S.9 Microscopy and Image Analysis

Ddx4 organelle portioning of fluorescent nucleic acids and proteins was imaged using a Leica TCS SP5II microscope equipped with a motorised stage and HCX PL APO CS 40× NA 1.3 oil immersion objective. Illumination was provided by Argon (458, 488 and 514 nm) and Helium-Neon (543 and 633 nm) lasers. Imaging scan speed was 400 Hz with a format of 512×512 pixels at 8 bit depth. Z-stacks were taken+/−10 μm from the brightest plane of the sample in 1 μm increments. Experimental parameters (laser intensity, detector sensitivity etc.) remained constant for each set of experiments and associated controls. Typical excitation and emission schemes for the fluorophores used in partitioning experiments are summarised in table 5. Image analysis was performed using Fiji³. Quantitation of partitioning was achieved by selecting circular regions of interest (ROIs) comprising 5 individual Ddx4 organelles (and 5 equally sized regions of adjacent dilute phase) for three fields of view (FOV) per sample with two independent samples per experiment. The z-stack for each ROI was cropped to 5 slices, centred on the middle of the sample (FIG. 6). The sum of the fluorescence from each ROI was divided by its volume to normalise for ROI size. The normalised fluorescence from Ddx4N1 protein-only control samples was used to baseline-correct samples containing fluorescent material. The ratio of the baseline-subtracted sum/vol for each organelle and nearest adjacent region of dilute phase was taken, and converted into a free energy via:

ΔGpart=−RT ln([in]/[out])

where [in] and [out] are the total concentrations of oligonucleotides inside and outside the organelles. The reported values are the mean and standard deviation of the partitioning of 30 organelles (5 per FOV, 3 FOVs per sample, 2 samples) per fluorescent oligo or protein.

TABLE 5
Summary of excitation and emission schemes of flurophores
used in this study.

| Fluoropho [symbol] | Excitation (nm) | Typical emission |
|---|---|---|
| CFP | 458 | 465-505 |
| GFP | 488 | 500-600 |
| YFP | 514 | 525-600 |
| Cy3 | 514 (543 in 3 [symbol] | 555-620 |
| Cy5 | 633 | 650-750 |

[symbol]

indicates data missing or illegible when filed

### S.10 Thermodynamic Analysis of Combined Partitioning (Δ_{Gpart}) and Changes in Structure (ΔGstab) S.10.1 Analysis of Single Stranded Oligonucleotide Destabilisation

The partitioning data of single stranded nucleic acids was analysed according to the following thermodynamic square (FIG. 3bi), where folding and unfolded forms are distinguished by f and u:

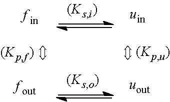

Which is defined by four equilibrium coefficients

$${K}_{S , o} = \frac{{f}_{out}}{{u}_{out}} , {K}_{S , i} = \frac{{f}_{i n}}{{u}_{i n}} , {K}_{P , f} = \frac{{f}_{out}}{{f}_{i n}} , {K}_{P , u} = \frac{{u}_{out}}{{u}_{i n}}$$

denoting the stability outside and inside the organelle (K_{S,o} and K_{S,i}) and the partition coefficient for folded and unfolded single stranded oligonucleotides (K_{P,f} and K_{P,u}). As before, due to the square geometry of the scheme:

K_{S,o}K_{P,u}=K_{S,i}K_{P,f}

The total concentration of oligonucleotide molecules is given by

ssₜₒₜ=fₒᵤₜ+fᵢₙ+uₒᵤₜ+uᵢₙ

If we substitute in equilibrium constants we solve for uₒᵤₜ:

$${u}_{out} = \frac{{K}_{P , u} {K}_{P , f} {ss}_{tot}}{( {K}_{P , f} + 1 ) {K}_{P , u} {K}_{S , o} + ( {K}_{P , u} + 1 ) {K}_{P , f}}$$

Our experimental observables are total concentration, stability of the DNA outside of the organelles, and the total partition coefficient are given by:

$$Δ {G}_{stab} = - RT ln \frac{{f}_{out}}{{u}_{out}} , {K}_{P}^{u} = \frac{{u}_{i n}}{{u}_{out}} , {K}_{P}^{obs} = \frac{{f}_{m} + {u}_{i n}}{{f}_{out} + {u}_{out}}$$

Where in the case of an unstructured single stranded oligonucleotide, ΔGpart=⁻RT ln KP^{u}, and for a structured single stranded oligonucleotide ΔGₚₐᵣt=−RT ln KP^{obs}. These can be manipulated to return to our original definitions:

$${K}_{S , o} = exp ( - Δ {G}_{stab} / RT )$$

$${u}_{out} = \frac{{ss}_{tot}}{( 1 + {K}_{P}^{obs} ) ( 1 + {K}_{S , o} )}$$

$${K}_{P , u} = \frac{1}{{K}_{P}^{u}}$$

$${K}_{P , f} = \frac{{K}_{S , o}}{{K}_{P}^{obs} ( {K}_{S , o} + 1 ) - {K}_{P}^{u}}$$

$${K}_{S , i} = \frac{{K}_{S , o} {K}_{P , u}}{{K}_{P , f}}$$

Finally, we define a destabilisation energy, the difference in free energy of DNA association inside and outside the organelles (FIG. 3c, hairpin data):

$$Δ Δ {G}_{destab} = Δ {G}_{stab}^{out} - Δ {G}_{stab}^{ i n} = - RT ln \frac{{K}_{S , o}}{{K}_{S , i}} = - RT ln \frac{{K}_{P , f}}{{K}_{P , u}} = - RT ln \frac{{K}_{S , o} {K}_{P}^{u}}{{K}_{P}^{obs} ( {K}_{S , o} + 1 ) - {K}_{P}^{u}}$$

Overall, from an experimental measurement of KP^{U}, KP obs, ΔGstab and knowledge of the total concentration ssTot, we can calculate the destabilisation energy for secondary structure in single stranded oligonucleotides.

### S.10.2 Analysis of Double Stranded Nucleotide Destabilisation

The analysis of double stranded oligonucleotides destabilisation is similar to that for the single strands (section S.10.1) but not identical as when a single chain denatures, we are left with one molecule, whereas when a duplex unfolds we have two. The partitioning data of double stranded nucleic acids was analysed according to the following thermodynamic square (FIG. 3bii):

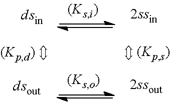

Which is defined by four equilibrium coefficients

$${K}_{S , o} = \frac{4 {ds}_{out}}{{ss}_{out}^{2}} , {K}_{S , i} = \frac{4 {ds}_{i n}}{{ss}_{i n}^{2}} , {K}_{P , d} = \frac{{ds}_{out}}{{ds}_{i n}} , {K}_{P , s} = \frac{{ss}_{out}}{{ss}_{i n}}$$

denoting the stability outside and inside the organelle (K_{S,o} and K_{S,i}) and the partition coefficient for double and single stranded oligonucleotides (K_{P,d} and K_{P,s}) The equilibrium constants are related to Gibb's free energies via ΔG=−RTlnK. As the geometry of the scheme is a square, any two sides are equivalent and so:

K_{S,o}K_{P,s}²=K_{S,i}K_{P,d}

The total concentration of oligonucleotide molecules is given by ssₜₒₜ=2dsₒᵤₜ+2dsᵢₙ+ssₒᵤₜ+ssᵢₙ. if we substitute in equilibrium constants we can express the total concentration in terms of just ssₒᵤₜ.

K_{S,o}K_{P,s}²=K_{S,i}K_{P,d}

The total concentration of oligonucleotide molecules is given by ssₜₒₜ=2dsₒᵤₜ+2dsᵢₙ+ssₒᵤₜ+ssᵢₙ. If we substitute in equilibrium constants we can express the total concentration in terms of just ssₜₒₜ.

$${ss}_{tot} = \frac{1}{2} {ss}_{out}^{2} {K}_{S , o} ( 1 + \frac{1}{{K}_{P , d}} ) + {ss}_{out} ( 1 + \frac{1}{{K}_{P , s}} )$$

Which can be solved:

$${ss}_{out} = - ( 1 + \frac{1}{{K}_{P , s}} ) + \sqrt{{( 1 + \frac{1}{{K}_{P , s}} )}^{2} + 2 {K}_{S , o} ( 1 + \frac{1}{{K}_{P , d}} ) {ss}_{tot}} / {K}_{S , o} ( 1 + \frac{1}{{K}_{P , d}} )$$

Our experimental observables are total concentration, stability of the DNA outside of the organelles, and the total partition coefficient are given by:

$$Δ {G}_{stab} = - RT ln \frac{4 {ds}_{out}}{{ss}_{out}^{2}} , {K}_{P}^{ss} = \frac{{ss}_{i n}}{{ss}_{out}} , {K}_{P}^{ds} = \frac{2 {ds}_{i n} + {ss}_{i n}}{2 {ds}_{out} + {ss}_{out}}$$

Where for a single stranded nucleotide ΔGₚₐᵣₜ=−RTlnK_{P}^{SS}, and for a double stranded, ΔGₚₐᵣₜ=−RTln K_{P}^{DS}. These can be manipulated to return to our original definitions:

$${K}_{S , o} = exp ( - Δ {G}_{stab} / RT )$$

$${ss}_{out} = ( - 1 + \sqrt{1 + 2 {K}_{S , o} {ss}_{tot} / ( 1 + {K}_{P}^{ds} )} ) / {K}_{S , o}$$

$${K}_{P , s} = \frac{1}{{K}_{P}^{ss}}$$

$${K}_{P , d} = \frac{{ss}_{out} {K}_{S , o}}{{K}_{P}^{ds} ( {ss}_{out} {K}_{S , o} + 2 ) - 2 {K}_{P}^{ss}}$$

$${K}_{S , i} = \frac{{K}_{S , o} {K}_{P , s}^{2}}{{K}_{P , d}}$$

Finally, we define a destabilisation energy, the difference in free energy of DNA association inside and outside the organelles:

$$Δ Δ {G}_{destab} = Δ {G}_{stab}^{out} - Δ {G}_{stab}^{ i n} = - RT ln \frac{{K}_{S , o}}{{K}_{S , i}} = - RT ln \frac{{K}_{P , d}}{{K}_{P , s}^{2}}$$

Overall, from experimental measures of KP^{SS}, KP^{DS}, ΔG_{stab} and sstot values, ΔG_{destab} for double stranded DNA can be directly calculated (FIG. 3c, double stranded DNA/RNA/hybrid data).

### S.11 Topological Frustration of Nucleic Acids Inside Membraneless Organelles

The per-residue thermodynamic quantities ΔGₚₐᵣₜ/N and ΔG_{destab}/N were observed to decrease as a function of length, suggesting that the contribution per residue decreases as a function of sequence length. This is to be expected where there is topological frustration in the system⁴. When a single strand is dissolved inside an organelle, each residue will make many weak interactions with itself and solvent. Individual residues in shorter oligonucleotides will have the freedom to orientate themselves most favourably, whereas for longer oligonucleotides this will be lower if the mobility of the molecule is restricted. This scaling, where the free energy per residue decreases as a function of length in this way can be considered a form of topological frustration. In general terms, a reduced description of the free energy can be given in terms of factors that depend on its volume, and those that depend on its surface area. The former are proportional to the radius cubed, and so depends on N. The latter depends on the radius squared, and so depends on N^{2/3}.

ΔG=volume+surface=aN+bN^{2/3}

The free energy per residue is then expected to have a particular variation with length:

$$\frac{Δ G}{N} = a + \frac{b}{{N}^{1 / 3}}$$

This is precisely the scaling seen in both DNA destabilisation (FIG. 3c, solid lines, FIG. 7c), and intrinsic single and double stranded DNA partitioning. The fitting parameters obtained are summarised in table 6. These results collectively show that individual bases within longer oligonucleotides contribute less than individual bases within shorter oligonucleotides, suggesting that the liquid drops are more able to restructure and stabilise shorter molecules than longer ones.

TABLE 6
Summary of the fitting parameters describing topological
frustration in free energies the system.

| | a | b |
|---|---|---|
| ΔG_{destab} | −2.13 +/− 0.26 | 7.85 +/− 0.75 |
| ΔGₚₐᵣₜ^{ss} | 0.81 +/− 0.09 | −3.20 +/− 0.25 |
| ΔGₚₐᵣₜ^{ds} | 2.43 +/− 0.60 | 0.84 +/− 0.21 |

### S.12 Duplex Destabilisation by Ddx4 Organelles Using FRET-Pair Nucleic Acids

A series of FRET samples based on the dsDNA or ssDNA 12mer ACTG oligonucleotides and 24mer dsDNA ACTG oligonucleotides were used to investigate whether Ddx4 organelles destabilised nucleic acid duplexes. Measurements were made using confocal microscopy (section S.12.1). The differently labelled oligonucleotides used in the study are listed in table 7, the correction factors measured in control measurements for a FRET calculation are shown in table 8 and the final results are summarised in table 9. To validate these results, FRET experiments were repeated in a fluorimeter (section S.12.2) in the presence of increasing amounts of the denaturant guanadinium hydrochloride (GdmHCl).

### S.12.1 Measurement of FRET by Confocal Microscopy

In each FRET experiment involving 12mer ssDNA and 12mer and 24mer dsDNA samples, 3 fluorescence images were recorded per z-slice. This produced three z-stacks corresponding to the following absorption/emission schemes:
i. DDobs=observed donor emission after donor excitation.
ii. DAobs=observed acceptor emission after donor excitation (FRET).
iii. AAobs=observed acceptor emission after acceptor excitation.

In FRET experiments containing only Cy3 and Cy5 fluorophores, the donor fluorophore (Cy3) was excited using a 514 nm laser and the acceptor fluorophore (Cy5) was excited using a 633 nm laser. Donor emission was collected between 555 and 620 nm, whilst acceptor emission was collected between 650 and 750 nm.

In FRET experiments involving 24mer ACTG dsDNA in the presence of GFP^{WT} or GFP⁺¹⁵: GFPs were excited using a 488 nm maser, Cy3 was excited using a 543 nm laser and Cy5 was excited using a 633 nm laser. Emission bands for Cy3 and Cy5 as above.

The overlap of acceptor and donor emission and excitation bands means that the observed emission values need to be corrected to obtain a FRET measurement. To account for this, equivalent emission intensities as above were recorded using samples that contained only donor (DD_{donor}, DA_{donor}) or acceptor fluorophores (DA_{accept}, AA_{accept}) with identical hardware parameters (i.e. laser intensity, detector sensitivity and emission bandwidth). Image processing and analysis was performed as described in section S.9. The volume-normalised and base-lined fluorescence intensities for equivalent ROIs in each of the DD_{obs}, DA_{obs}, AA_{obs}, DD_{donor}, DA_{donor}, DA_{accept} and AA_{accept} image stacks were recorded and used for subsequent calculations. The corrected FRET, E_{CT}, is then obtained from the following:

$${E}_{ct} = \frac{DA}{DA + DD} = \frac{{DA}_{obs} - α \cdot {DD}_{obs} - β \cdot {AA}_{obs}}{{DD}_{obs} + ( {DA}_{obs} - α \cdot {DD}_{obs} - β \cdot {AA}_{obs} )}$$

where the correction terms:

$$α = \frac{{DA}_{donor}}{{DD}_{donor}} and β = \frac{{DA}_{accept}}{{AA}_{accept}}$$

Measurements were performed on single stranded and double stranded 12mers, inside and outside organelles, and in dilute solution where no organelles were present. The correction factors and FRET measurements are summarised in table 8. FRET was calculated on a per-ROI (organelle or region of dilutes phase) basis. The reported FRET values are the mean and standard deviation of the corrected FRET signals of 30 organelles or equivalently sized regions of dilute phase (5 per field of view (FOV), 3 FOVs per sample, 2 samples).

For the FRET images in FIG. 2aiii and FIG. 4bii, representative organelles were selected, and the frame cropped in the xy plane to include some of the surrounding dilute region. Next, the z-stack was cropped to the 5 slices at the middle of the sample. The same procedure was applied to each of the three fluorescence z-stacks recorded in the FRET experiment. A z-projection of the sum of intensities was next produced for each image stack, and a corrected FRET_{(Ect)} composite image was generated according to the E_{ct} equation above, with the appropriate z-projection images substituted for the DDobs, DAobs and AAobs terms. The average of the inside and outside organelle correction factors (α and β, table 8, FIG. 8c) were used to scale the whole images. The mean grey values obtained for the organelle region of the processed images were subsequently linearly scaled to those obtained after the full analysis of many organelles, fields of view and samples so that the images gave a representative visualisation of the data.

TABLE 7
Summary of constructs used for the FRET
experiment (FIG. 2aiii, and FIG. 4bii). No
significant differences in partitioning were
observed for these constructs when compared
to those investigated in FIG. 1c.

| | | | sequence |
|---|---|---|---|
| | Length | sequence | (additional |
| label | (bp) | (sense strand) | strand) |
| dsDNA_ | 12 | [Cy5] | CAGTCAGTCAGT[Cy3] |
| ACTG_ | | ACTGACTGACTG | (SEQ ID NO: 13) |
| FRET | | (SEQ ID NO: 12) | |
| dsDNA_ | 12 | ACTGACTGACTG | CAGTCAGTCAGT[Cy3] |
| ACTG_ | | (SEQ ID NO: 12) | (SEQ ID NO: 13) |
| DONOR | | | |
| dsDNA_ | 12 | [Cy5] | CAGTCAGTCAGT |
| ACTG_ | | ACTGACTGACTG | (SEQ ID NO: 13) |
| ACCEPTOR | | (SEQ ID NO: 12) | |
| ssDNA_ | 12 | [Cy5] | ACTGACTGACTG[Cy3] |
| ACTG_ | | ACTGACTGACTG | (SEQ ID NO: 12) |
| FRET | | (SEQ ID NO: 12) | |
| ssDNA_ | 12 | ACTGACTGACTG | ACTGACTGACTG[Cy3] |
| ACTG_ | | (SEQ ID NO: 12) | (SEQ ID NO: 12) |
| DONOR | | | |
| ssDNA_ | 12 | [Cy5] | ACTGACTGACTG |
| ACTG_ | | ACTGACTGACTG | (SEQ ID NO: 12) |
| ACCEPTOR | | (SEQ ID NO: 12) | |
| dsDNA_ | | [Cy5] | CAGTCAGTCAGTCAGTC |
| ACTG_ | 24 | ACTGACTGACTGA | AGTCAGT[Cy3] |
| FRET | | CTGACTGACTG | (SEQ ID NO: 19) |
| | | (SEQ ID NO: 35) | |
| dsDNA_ | 24 | ACTGACTGACTGA | CAGTCAGTCAGTCAGTC |
| ACTG_ | | CTGACTGACTG | AGTCAGT[Cy3] |
| DONOR | | (SEQ ID NO: 35) | (SEQ ID NO: 19) |
| dsDNA_ | 24 | [Cy5] | CAGTCAGTCAGTCAGTC |
| ACTG_ | | ACTGACTGACTGA | AGTCAGT |
| ACCEPTOR | | CTGACTGACTG | (SEQ ID NO: 19) |
| | | (SEQ ID NO: 35) | |

TABLE 8
Summary of correction factors and FRET measurements for double and
single stranded DNA, inside, outside and in the absence of organelles
from confocal microscopy measurements.

| | | | | | Organelle | |
|---|---|---|---|---|---|---|
| | Outside | err | inside | err | free | err |
| α (ss 12mer) | 0.434 | 0.06 | 0.433 | 0.053 | 0.05 | 0.005 |
| α (ds 12mer) | 0.184 | 0.022 | 0.197 | 0.027 | 0.046 | 0.003 |
| α (ds 24mer) | 0.063 | 0.008 | 0.069 | 0.014 | | |
| 13 (ss 12mer) | 0.182 | 0.030 | 0.167 | 0.028 | 0.011 | 0.001 |
| 13 (ds 12mer) | 0.029 | 0.005 | 0.027 | 0.006 | 0.012 | 0.001 |
| 13 (ds 24mer) | 0.066 | 0.008 | 0.062 | 0.011 | | |
| EC-(ss 12mer) | 0.029 | 0.065 | 0.122 | 0.061 | 0.068 | 0.038 |
| EC-(ds 12mer) | 0.778 | 0.053 | 0.52 | 0.035 | 0.73 | 0.031 |
| EC-(ds 24mer) | 0.742 | 0.025 | 0.524 | 0.036 | | |
| α (ds 24mer+ | 0.057 | 0.006 | 0.064 | 0.011 | | |
| α (ds 24mer+ | 0.064 | 0.009 | 0.08 | 0.01 | | |
| 13 (ds 24mer+ | 0.061 | 0.007 | 0.049 | 0.01 | | |
| 13 (ds 24mer+ | 0.156 | 0.023 | 0.144 | 0.02 | | |
| EC-(ds 24mer+ | 0.784 | 0.022 | 0.591 | 0.031 | | |
| EC-(ds 24mer+ | 0.651 | 0.04 | 0.341 | 0.05 | | |

### S.12.2 Measurement of FRET by Fluorimeter

Fluorescence measurements were made of the FRET-pair ssDNA and dsDNA 12mer ACTG samples in the absence of organelles but in the presence of increasing concentrations of the denaturant GdmHCl using a Perkin Elmer LS-50B fluorescence spectrometer. The 12mer ACTG dsDNA FRET-pair was chemically denatured using guanidinium chloride (GdmHCl 0-6 M, pH 8.0). At each titration point the total concentration of DNA was 2 tM (i.e. sense and sense/antisense strands each at 1 tM), and the NaCl concentration was fixed at 150 mM.

For samples not containing GdmHCl, Cy3 (donor) and Cy5 (acceptor) excitation was achieved at 514 and 633 nm, respectively, each with slit widths of 7.5 nm. Spectra (500 and 800 nm) were collected with a scan rate of 240 nm min⁻¹. For samples containing GdmHCl, Cy3 (donor) and Cy5 (acceptor) excitation was achieved at 514 and 633 nm, respectively, each with slit widths of 5 nm. Spectra (500 to 800 nm) were collected with a scan rate of 120 nm min¹.

The data were quantified using the integrative ratio A (RA) method⁵. The spectrum (intensity values are a function of emission frequency w) was recorded after donor excitation for samples containing donor only f(D,w)donor and a mixture of donor and acceptor f(D,w)ₘiₓ, and after acceptor excitation for the mixture f(A,w)ₘiₓ. The ratios were then calculated as follows:

$${R}_{A} = \frac{{\sum }_{ω} {f ( D , ω )}_{mix} - {Nf ( D , ω )}_{donor}}{{\sum }_{ω} {f ( A , ω )}_{mix}} where N = \frac{{f ( D , max )}_{mix}}{{f ( D , max )}_{donor}}$$

Where max is the value of w where the donor emission is a maximum. The values of

RA were found to decrease as GdmHCl concentration was increased in the double stranded DNA samples, indicating destabilization of the double strand with denaturant (summarised in table 9). RA data was linearly scaled to the corrected FRET values obtained from equivalent samples using confocal microscopy, with ssDNA and dsDNA (no denaturant) as lower and upper bounds, respectively (FIG. 2c).

TABLE 9
A summary of RA values
obtained as a function of GdmHCl concentration.

| | [GdmHCl] (mol | |
|---|---|---|
| | dm⁻³) | RA |
| ssDNA | 0 | 0.02940 |
| dsDNA | 6 | 0.18077 |
| dsDNA | 5 | 0.27513 |
| dsDNA | 4 | 0.30104 |
| dsDNA | 3 | 0.40341 |
| dsDNA | 0.3 | 0.42226 |
| dsDNA | 0 | 0.44761 |

TABLE 10
Ddx4 protein variants used in pH solubility experiment.
PTM stands for post-translational modification.

| | residue | | |
|---|---|---|---|
| Ddx4 proteins | number | Mutation | PTM |
| WT | 1-236 | — | — |
| aDMA-modified | 1-236 | — | 5-6 |
| | | | aDMA |
| Δ132-136 → D | 1-236 | deletion | — |
| 9F to A | 1-236 | Phe to Ala | — |
| Charge | 1-236 | charge redistribution | — |
| scrambled | | | |
| DEAD → YFP | 1-724 | heliase domain replaced with | — |
| | | YFP | |

### SUPPLEMENTARY REFERENCES

1. Nott, T. J. et al. Phase Transition of a Disordered Nuage Protein Generates Environmentally Responsive Membraneless Organelles. Mol Cell 57, 936-947, (2015).
2. Markham, N. R. & Zuker, M. UNAFold: software for nucleic acid folding and hybridization. Methods in molecular biology 453, 3-31, (2008).
3. Schindelin, J. et al. Fiji: an open-source platform for biological-image analysis. Nature methods 9, 676-682, (2012).
4. Baldwin, A. J. et al. Metastability of Native Proteins and the Phenomenon of Amyloid Formation. J Am Chem Soc 133, 14160-14163, (2011).
5. Clegg, R. M. Fluorescence Resonance Energy-Transfer and Nucleic-Acids. Method Enzymol 211, 353-388, (1992).

1. A method of selective separation of target molecules from a heterogeneous mixture of target molecules and non-target molecules in a solution, the method comprising:
providing a proteinaceous phase in the solution, wherein the proteinaceous phase comprises a matrix of self-assembling proteins, and
wherein the proteinaceous phase selectively absorbs the target molecules into the matrix and/or selectively excludes non-target molecules from the matrix thereby selectively isolating the target molecules from the non-target molecules in the solution.

2. The method according to claim 1, wherein the proteinaceous phase is bi-phasic.

3. The method according to claim 1 or 2, wherein the proteinaceous phase is provided in the solution by providing the self-assembling proteins in liquid phase in the solution, and allowing or inducing phase transition by the assembly of the self-assembling proteins into a matrix to form the proteinaceous phase.

4. The method according to any preceding claim, wherein the proteinaceous phase forms a globule.

5. The method according to claim 3 or claim 4, wherein the induction of the assembly of the self-assembling protein into the proteinaceous phase comprises the induction of a phase transition of the self-assembling protein.

6. The method according to any of claims 3 to 5, wherein the induction of the phase transition into the proteinaceous phase comprises modulation of temperature.

7. The method according to any of claims 3 to 5, wherein the induction of the phase transition into the proteinaceous phase comprises modulation of ionic strength.

8. The method according to any of claims 3 to 5, wherein the induction of the phase transition into the proteinaceous phase comprises modulation of pH.

9. The method according to any of claims 3 to 5, wherein the induction of the phase transition into the proteinaceous phase comprises modulation of the self-assembling protein concentration.

10. The method according to any preceding claim, wherein the self-assembling protein comprises repeating 8-10 residue blocks of alternating net charge.

11. The method according to any preceding claim, wherein the self-assembling protein comprises an over-representation of FG, GF, RG, and GR motifs within the positively charged blocks.

12. The method according to any preceding claim, wherein self-assembling protein comprises FG and GF pairs spaced by 8-11 residues apart and RG and GR pairs to spaced 4 residues apart.

13. The method according to any preceding claim, wherein the self-assembling protein comprises a protein derived from nuage, nuclear bodies, nuclear speckles, the spliceosome, the nucleolus, Cajal bodies, P-bodies or stress granules.

14. The method according to any preceding claim, wherein the self-assembling protein comprises Ddx4 protein, or fragments or variants thereof.

15. The method according to any preceding claim, wherein the self-assembling protein is selected from any one of the proteins listed in Table 1, or a variant, truncation, elongation, or homologue of a protein selected from any one of the proteins listed in Table 1.

16. The method according to any preceding claim, wherein the self-assembling protein comprises any one of EWSR1; LSM14A; NUCL; KHBRDS1; DDX4; DDX3X; RBM3; and EIF4H; or variants or homologues thereof; or combinations thereof.

17. The method according to any preceding claim, wherein the self-assembling protein is modified by mutation or post-translational modification in order to control phase transition properties and/or target molecule absorption properties of the proteinaceous phase.

18. The method according to any preceding claim, wherein the proteinaceous phase selectively excludes one or more, or all non-target molecules.

19. The method according to any preceding claim, wherein the target molecule comprises a biomolecule, a small molecule, natural or synthetic polymer, sugar chain, or fatty acid chain; or combinations thereof.

20. The method according to any preceding claim, wherein the target molecule comprises single stranded nucleic acid.

21. The method according to any preceding claim, wherein the target molecule comprises nucleic acid with a secondary structure, such as hairpin structure.

22. The method according to any of claims 1 to 19, wherein the target molecule comprises a protein having a partitioning Gibbs free energy (ΔGₚₐᵣₜ) of greater than 0 kJ mol⁻¹, resulting in net absorption.

23. The method according to any preceding claim, wherein the self-assembling protein is a chaperone for separation/isolation of another molecule for separation by linking thereto prior to phase transition into the proteinaceous phase.

24. The method according to any preceding claim, wherein the target molecule is a chaperone for separation/isolation of another molecule for separation.

25. The method according to claim 24, wherein the molecule for separation is linked to the target molecule chaperone.

26. The method according to claim 24 or 25, wherein the target molecule is a poly-aromatic molecule, such as a fluorescein or Texas Red, and the molecule for separation is tagged with the poly-aromatic molecule.

27. The method according to any preceding claim, wherein the heterogeneous mixture comprises or consists of cell or tissue extract.

28. The method according to any preceding claim, wherein the heterogeneous mixture is a bodily fluid sample.

29. A method of selective separation of target molecules from a heterogeneous mixture of target molecules and non-target molecules in a solution, the method comprising:
tagging the target molecule with a self-assembling protein capable of assembling into a proteinaceous phase, wherein the proteinaceous phase comprises a matrix of the self-assembling proteins;
inducing the assembly of the self-assembling protein into the proteinaceous phase,
wherein the tagged target molecules are internalised into the matrix of the proteinaceous phase during the assembly thereby selectively isolating the target molecules from the non-target molecules in the solution.

30. The method according to any preceding claim, wherein following absorption of the target molecule, the method further comprises the step of separating/isolating the proteinaceous phase from the solution.

31. The method according to any preceding claim, wherein the proteinaceous phase is provided within a cell (or population of cells).

32. A method of Ion Torrent Sequencing of RNA, wherein RNA extraction is provided by isolating the RNA from a solution according to the method of any of claims 1 to 31.

33. A composition comprising self-assembling proteins, wherein the self-assembling proteins are capable of assembling into a matrix to form a proteinaceous phase.

34. The composition according to claim 33, wherein the self-assembling proteins are in a reversible amorphous solid (glassy) state.

35. A kit for selective separation and/or modification of target molecules from a heterogeneous mixture of the target molecules and non-target molecules in a solution, wherein the kit comprises:
the self-assembling proteins described herein, which are capable of assembling into a matrix in solution to form a proteinaceous phase.

36. The kit according to claim 35, wherein the self-assembling proteins are provided in a reversible amorphous solid (glassy) state.

37. Use of self-assembling protein to selectively isolate target molecules from a heterogeneous mixture of the target molecules and non-target molecules in a solution.

38. A method, composition, or use substantially described herein, optionally with reference to the accompanying figures.
